(19) United States
(12) Patent Application Publication
(10) Pub. No.: US 2021/0137549 A1
(43) Pub. Date: May 13, 2021

(54) DEVICE FOR CHEMICALLY ASSISTED DISSECTION

(71) Applicant: AuXin Surgery SA, Louvain-la-Neuve (BE)

(72) Inventors: Gilles Capart, Brussels (BE); Benoît Verjans, Louvain-la-Neuve (BE); Laurent Ferrière, Louvain-la-Neuve (BE)

(21) Appl. No.: 17/111,265

(22) Filed: Dec. 3, 2020

Related U.S. Application Data
(63) Continuation-in-part of application No. 16/118,928, filed on Aug. 31, 2018.
Application No. 16/118,928 is a continuation of application No. 14/889,084, filed on Nov. 4, 2015, now Pat. No. 10,092,312.

(30) Foreign Application Priority Data
May 7, 2013 (EP) 13166898.0
Dec. 19, 2013 (EP) 13198443.7

(51) Int. Cl. A61B 17/3203; A61M 5/24; A61M 5/14

(52) U.S. Cl. CPC A61B 17/3203; A61B 2090/0816; A61M 5/1407; A61M 5/2448

(57) ABSTRACT

A device and a method for delivering a solution to tissues and/or organs. The solution contains at least one solvent and at least one solute. The device includes at least one chamber which contains sodium 2-mercaptoethanesulfonate (Mesna) in powder form. The method includes dissolving the solute in the solvent inside the device. The resulting solution is delivered immediately to tissues and/or organs to facilitate dissection. A composition including Mesna and one or more dye which is used in a method of surgery, a kit including Mesna, and a method for visualization of a composition including Mesna which is applied to tissues. A method for producing a composition including Mesna for use in a method of surgery, a kit for obtaining a composition including Mesna for use in a method of surgery, a device for chemically assisted tissue dissection facilitated by Mesna, and a method of surgery using Mesna.

### RELATED APPLICATIONS

This application is a continuation-in-part of U.S. patent application Ser. No. 16/118,928, filed Aug. 31, 2018, which is a continuation of U.S. patent application Ser. No. 14/889,084 (now U.S. Pat. No. 10,092,312), filed Nov. 4, 2015, which is the U.S. National Phase under 35 U.S.C. 371 of PCT/EP2014/059332, filed May 7, 2014, which claims priority to European Patent Application No. 13166898.0, filed May 7, 2013, and European Patent Application No. 13198443.7, filed Dec. 19, 2013, the entire contents of which are incorporated herein by reference.

### FIELD OF THE INVENTION

The present invention relates to a device and a method for delivering a solution to biological tissues. In particular, the invention relates to devices for mixing and delivering chemicals for biological tissues separation and/or assisting said separation.

The present invention further relates to a composition comprising sodium 2-mercaptoethanesulfonate (referred to herein as “Mesna”), for use in a method of surgery, a kit comprising Mesna and a method for visualization of a composition including Mesna which is applied to tissues.

The present invention further relates to a composition comprising Mesna for use in a method of robot-assisted surgery, to a method for producing a composition including Mesna for use in a method of robot-assisted surgery, to a kit for obtaining a composition including Mesna for use in a method of robot-assisted surgery, to robot instruments for chemically assisted tissue dissection facilitated by Mesna, and to a method of robot-assisted surgery using a composition including Mesna.

### BACKGROUND

Dissection instruments have found ample usage as manual surgical instruments in multiple surgical fields including abdominal surgery, orthopedics and neurosurgery. Their action is to mechanically dissect tissues. Dissectors assist in the detachment of normal tissues to enable appropriate penetration through the soft tissue layers or in the detachment of various pathological tissues from healthy tissues.

The detachment of tissues using standard mechanical tissue dissectors can be quite tedious and is often responsible for long surgical operations. In addition, post-operative complications may occur in some surgical procedures such as impairing the function of the remaining tissues, nerves or veins, hemorrhage, infections and recurrence. These complications result from the difficulties that surgeons may encounter in highlighting the cleavage planes, detaching tissue layers and obtaining hemostasis. An important technical progress in this field would be to facilitate the detachment of tissues, increase the success rate of cyst or tumor removals, avoid damage of remaining surrounding organs, reduce blood loss and post-operative adhesions, thereby reducing operation duration, reduce medical costs of such operations as well as health risks for patients.

When applied at the cleavage plane, Mesna breaks the molecular bonds between tissue layers, thereby facilitating tissue separation. Specifically, Mesna breaks disulfide bonds of polypeptide chains and proteins. Accordingly, Mesna weakens inter-tissues and/or organs adhesion to facilitate dissection along said cleavage plane.

However, nowadays for tissue separation, Mesna is only available in liquid form. A major drawback of Mesna solutions is their instability as mentioned in U.S. Pat. No. 5,728,738. The liquid form is highly prone to oxidation and is therefore highly unstable especially in presence of metals. Therefore, it is common to store Mesna solutions in low iron glass containers under nitrogen blanket with stabilizers and anti-oxidants. When being used, the practitioner has to transfer the solution from glass containers to a delivery device or to a tube in order to bring the solution in contact with the desired tissue. This step increases contamination risk at the dissection site and increases surgery time. This is in addition to the high chances of oxidation of the stored Mesna solution thereby having a reduced Mesna activity when used for assisting surgery.

Other drawback of the Mesna in liquid form which is available today resides in the absence of concentration choice. In some procedures, larger quantities and/or different concentration of Mesna, from that readily available in commercial glass vials, are needed Indeed the practitioner can only dilute the Mesna which is available in liquid form and cannot use higher Mesna concentrations if required. This makes making the use of said vials tedious or inadequate. As mentioned in U.S. Pat. No. 5,728,738, Mesna solutions available are stabilized by addition of pH adjustment agents, additives such antioxidants and stabilizing agents thereby avoiding oxidation and/or the degradation of Mesna when the Mesna solution is stored. Addition of such agents presents a considerable risk for patient's health and increases the cost of said Mesna solutions. In addition, commercial solutions are not isotonic and may damage exposed cells when applied topically.

One of the objectives of the invention is to overcome at least part of the above mentioned problems. The invention aims at providing a device and method for increasing the shelf life of Mesna before use, optimizing the stability of the delivered Mesna in liquid form and for optimizing the delivery of said Mesna in liquid form to the target location at the desired concentration, volume and quality for surgical applications. In addition, the present invention aims at providing the user with a possibility to choose the concentration of the Mesna solution to be used. In addition, the invention aims at lowering the costs of the Mesna solution provided to the user.

U.S. Pat. No. 6,143,797 discloses methods of facilitating detaching pathological tissues from healthy tissues with which they have formed adherences and facilitating detachment of healthy tissues from other healthy tissues such as in plastic surgery in which an effective amount of Mesna is applied to the tissue or tissues.

This patent also discloses the problem of not specifying means of monitoring parts of tissues on which Mesna is effectively applied which could lead to inefficient application of Mesna on tissues in terms of inefficiencies in the quantity of Mesna and/or inefficiencies in the locations where Mesna is applied.

According to further embodiments, the present invention aims to resolve at least aforementioned problem regarding inefficient application of Mesna.

The further embodiments of the invention thereto aim to provide a composition including Mesna, wherein the composition further comprises one or more dyes.

There remains a need in the art for improvements in surgery, e.g. in terms of reducing health risks for patients and providing tools for aiding medical practitioners in performing surgery.

Many surgical procedures involve dissection steps where pathologic material must be separated from critical organs, such as nerves, muscles, ducts, ovaries, veins, etc., without damaging such organs. In those situations, the use of non-selective dissection tools, such as electric (RF) knives or laser knives may damage irreversibly these organs and cause surgery side effects.

On the other hand, non-cutting mechanical dissectors may fail to separate strongly adhering tissues or tear or damage the organs to be preserved.

The use of robots in surgery, which are normally fitted with electric knives and mechanical dissectors can improve the precision of dissections but, in general would not result in a significant reduction of surgery side effects.

Embodiments of the invention target at solving at least one of the aforementioned disadvantages in surgery.

### SUMMARY

The present invention provides a device for delivering a solution comprising at least one solvent and at least one solute. Preferably, the solution consists of one solute and one solvent. The solute is a mucolytic agent in powder form. Said mucolytic agent is sodium 2-mercaptoethanesulfonate, known as Mesna, in powder form. Preferably, the device comprises at least one chamber which comprises sodium 2-mercaptoethanesulfonate in powder form. Preferably said solvent is a sterile physiological saline solution or saline water wherein the content of NaCl is adjusted to make the solution isotonic. The use of isotonic solution in surgery is advantageous as it prevents the exposed cells in the surgery site from damage.

In a preferred embodiment, the device comprises a first chamber for housing the solute, a second chamber for housing the solvent and at least one outlet suitable to be in fluid communication with at least one of the chambers; said chambers are separated from each other by at least one disruptable separation means and are in fluid communication with each other upon disruption of said separation means thereby forming the solution. In a preferred embodiment, said device comprises at least one pressure means for manually applying a pressure on the first chamber walls and/or the second chamber walls thereby delivering the solution to said tissues and/or organs. Preferably, the wall of the first chamber walls and/or the second chamber is made of a flexible material.

In a preferred embodiment, the separation means comprises a spatial separation between the first chamber and the second chamber. Said separation means can also be devoid of spatial separation between the first chamber and the second chamber. Preferably, the separation means comprises at least one disruption means for disrupting said separation means.

In a preferred embodiment, the device comprises a single chamber for housing the solute, at least one outlet suitable to be in fluid communication with the single chamber and at least one connection means which is in fluid communication with said single chamber thereby connecting the single chamber to a solvent source.

The device according to any embodiment of the invention is connectable to and/or controlled by an electrically driven mechanical system for the delivery of Mesna solution to the target location.

In a preferred embodiment, the device consists of a single chamber for housing the solute. The device is provided with at least one outlet and is suitable to be connected—via said outlet—to a solvent reservoir in order to produce a solution that can be used with high pressure liquid delivery device. Said solvent is preferably saline water wherein the NaCl is adjusted to make said solution isotonic. For instance, the solvent reservoir is connectable to a solvent jet surgery system known to the person skilled in the art as water jet surgery system. The reservoir with the Mesna solution replaces the physiological water bag present in conventional known water jet surgery systems, also called hydrosurgery.

The device according to the invention is suitable to be used as a chemically assisted tissue dissector instrument. Said device is suitable to be connected to any surgical dissector known to the person skilled in the art provided with fluid connection for dispensing at the active edge of said surgical dissector. The device allows the topical and local instillation of a chemical solution to facilitate the mechanical dissection and separation of tissue.

In a preferred embodiment, the device comprises at least one air vent for air evacuation of the device and/or air insertion into the device. The device is designed to be hand held and hand manipulated by the operator.

The invention further provides a kit comprising sodium 2-mercaptoethanesulfonate is in powder form, a solvent for dissolving said sodium 2-mercaptoethanesulfonate and a device according to any of the preceding claims wherein said device comprises at least said sodium 2-mercaptoethanesulfonate is in powder form. In a preferred embodiment, the solvent is saline water wherein the content of NaCl is adjusted to make the solution isotonic.

The present invention further provides a method for weakening inter-tissues and/or organs adhesion to facilitate dissection by delivering a solution comprising at least one solvent and at least one solute to tissues and/or organs. Preferably, the solution consists of one solute and one solvent. The method comprises the steps of dissolving said solute in said solvent thereby obtaining the solution and delivering the obtained solution to said tissues and/or organs; said solute is a mucolytic agent in powder form. The solution is preferably immediately delivered after being obtained. Preferably, said mucolytic agent is sodium 2-mercaptoethanesulfonate also known as Mesna. More preferably Mesna is used in powder form. Preferably said solvent is a sterile physiological saline solution wherein the content of NaCl is adjusted to make the solution isotonic.

Immediately delivered after being obtained means that the solution is delivered at most 24 hours, preferably at most 12 hours, more preferably at most 6 hours, even more preferably at most 4 hours, most preferably at most 30 min, even most preferably at most 10 min after dissolving the solute in the solvent. In a further preferred embodiment, said solution is delivered at most 5 min, preferably at most 4 min, more preferably at most 3 min, most preferably immediately, i.e. 0 min, after dissolving the solute in the solvent. Preferably, the solution is solution is delivered in droplets form having a predetermined volume.

In a preferred embodiment, the solute and/or the solvent are devoid of antioxidants and/or stabilizing agents.

In one embodiment, the method further comprises the steps of providing a device comprising two chambers as described above, disrupting the separation means of the device thereby obtaining the solution, and delivering the obtained solution through the outlet of the device to a target location.

In another embodiment, the method comprises the steps of providing a device comprising a single chamber as described above, connecting said device and more in particular said single chamber to an external solvent source, inserting the solvent into said single chamber thereby obtaining the solution, i.e. Mesna solution, and delivering the obtained solution through the outlet of the device to a target location. Preferably, the solution consists of one solute and one solvent.

In another embodiment, the method comprises the steps of providing a device comprising a single chamber as described above suitable to be connected to a reservoir containing a solvent, inserting the solute into said reservoir thereby obtaining the solution, i.e. Mesna solution, connecting said reservoir containing said Mesna solution to a water jet surgery system, and delivering the obtained solution to a target location.

Preferably, the solution consists of one solute and one solvent. Said delivery is made through a delivery means of water jet surgery system thereby, delivering Mesna solution to the target location at high pressure. Said delivery means is selected from the group comprising: a surgical device, a high pressure pump, a delivery tube, an applicator or any combination thereof.

The invention further provides for the use of a mucolytic agent in hydrosurgery. Said mucolytic agent is sodium 2-mercaptoethanesulfonate, preferably in powder form. The invention further provides hydrosurgery method comprising the steps of: connecting a first container comprising the solute to a second container comprising the solvent;

transferring at least partially the content of the first container into the second container thereby dissolving the solute in the solvent and obtaining the solution; disconnecting the first container from the second container, and delivering the obtained solution to said tissues and/or organs, wherein said solute is sodium 2-mercaptoethanesulfonate in powder form.

The invention also provides a kit comprising at least one first container which is sealed by a disruptable membrane and comprising the solute, at least one second container comprising the solvent and at least one disrupting device for disrupting the disruptable membrane of the first container wherein said solute is sodium 2-mercaptoethanesulfonate in powder form. In a preferred embodiment, the solvent is saline water wherein the content of NaCl is adjusted to make the solution isotonic. The use of isotonic solution in surgery is advantageous as it prevents the exposed cells surrounding the surgery site from damage.

The device of the invention is connectable to dissection instruments, via a luer lock fluid connection for instance. The device enables the controlled instillation of the synthetic sulfur compound Mesna during tissue dissection directly in contact with the tissue to be detached thus reducing its diffusion. Mesna facilitates the mechanical tissue separation by the surgical dissector. The chemical compound for instillation is located in the device and thus forms an integral part of the device. Mesna is stored in the device in powder form before use to take advantage of the superior stability properties of the solid powder form compared to the formulated solution.

The device and the method of the invention present several advantages. Oxidation of Mesna solution before delivery is avoided as said solution is prepared immediately before said delivery. Another advantage is the absence of antioxidants and/or stabilizing agents, the use of which is not required as the solution is prepared immediately prior use. This reduces the risk of potential side effects from the use of such additives and the cost of the prepared solution. The invention further provides a maximized sterility of the delivered solution and thereby of the surgery and/or the treatment. In addition, the invention provides for a controlled delivery of the Mesna solution which can be delivered at the practitioner's will and at the required time. It avoids cumbersome manipulations of solutions from glass vials in order to obtain the desired concentration and volume. Furthermore, the invention offers to the user the possibility to choose the concentration of Mesna solution to be used.

The device and the method of the invention are easy to use and facilitate tissue dissection while preserving healthy tissue and organ functions. Observable benefits are to reduce the damage to the remaining tissues or organs, a reduction of pre- and post-operative bleeding, a reduction of post-operation adhesions, a reduction in surgical procedure time and an increased surgeon satisfaction. In addition, the invention allows decreasing hospital stay duration, preventing post-operative complications and allows decrease disease recurrence. Another advantage provided by the invention is the easy use and handling of the device. Said device is inexpensive and simple to produce.

The invention further provides a composition comprising Mesna, wherein the composition further comprises one or more dyes.

In particular, the composition comprising Mesna and one or more dyes is of use in surgery, and more in particular in surgery where Mesna is applied at a cleavage plane between two or more tissues for facilitating tissue separation. The dyes function to stain and, consequently, visualize parts of the tissues on which the Mesna composition is applied. This entails the advantage that a medical practitioner can see the parts where the composition is applied, allowing intuitive and quick ascertaining of a desired application of Mesna to the tissues, or if the application of Mesna should be adjusted, which adjustments can also be directly seen by the visualization brought about by the one or more dyes. The use of a dye allows to visualize whether the mesna solution remains in place for a sufficient duration for effective chemical action. It also helps visualize the cleavage plane, which is very useful to the surgeon. This is of great importance for allowing efficient dissection of tissues facilitated by Mesna, and certainly in view of the knowledge that flooding a surgical cavity in order to make sure that all tissues to be dissected are wetted by Mesna is not an option because the amount of Mesna which can be dispensed in a single surgical procedure is limited by toxicity considerations.

Preferred embodiments of the composition are provided.

The invention further provides a composition comprising Mesna, wherein the composition further comprises one or more dyes, for use in a method of surgery. Preferred embodiments thereof are given.

The invention further provides a kit comprising Mesna, wherein the kit further comprises one or more dyes. Preferred embodiments of the kit are provided.

The invention further provides a method for visualization of a composition comprising Mesna which is applied to tissues, comprising the step of applying the composition comprising Mesna to two or more tissues, wherein the composition further comprises one or more dyes for visualizing parts of the two or more tissues where the composition has been applied. Preferred embodiments of the method are provided.

The invention further provides a use of a composition comprising Mesna, wherein the composition further comprises one or more dyes, in said method for visualization.

Further embodiments of the invention aim to provide a composition comprising Mesna for use in a method of robot-assisted surgery and the robot instruments adapted to such use.

The invention further provides a composition comprising Mesna for use in a method of robot-assisted surgery.

A composition comprising Mesna for use in a method of robot-assisted surgery shows the advantages that the surgery is facilitated by breakage of molecular bonds between tissue layers by topical application of Mesna to a targeted cleavage plane between the tissue layers, while at the same time offering the precision and minimally invasive approach of a robot-assisted surgery. Moreover, when applying Mesna, cleavage planes between tissues are revealed. Accordingly, selective mechanical detachment of pathological tissues is possible without cutting. The application of Mesna also facilitates the dissection of fibrosis, which is very difficult by other means. This advantageous manner of mechanical detachment of tissues following breakage of molecular bonds by Mesna can be construed as chemically assisted tissue dissection. A method of surgery without the use of composition comprising Mesna would require more effort in separating tissue layers from another, resulting in a longer duration of surgery or even undesired tissue damage. In particular, without the use of composition comprising Mesna, dissection would have to be performed by electric or mechanical knives with increased risk of cutting accidentally or unavoidably critical organs.

Preferred embodiments of the composition comprising Mesna for use in a method of robotic assisted surgery are provided.

The compositions comprising Mesna without added colorants are colorless and the topical deposition of the solution to tissues to be dissected is hardly visible by the robot operator. The addition of a dye is a preferred embodiment. As a further embodiment, the dye is visible in fluorescence for further discrimination of the tissues to be dissected.

The invention further provides a method for producing a composition comprising Mesna for use in a method of robot-assisted surgery.

The measure of preparing the composition by dissolving Mesna in powder form in one or more pharmaceutically acceptable solvents and/or one or more pharmaceutically acceptable buffers, at most one day prior to performing the method of surgery, has the advantage that the prepared composition comprising Mesna can be used shortly after dissolving Mesna in powder form, thereby providing maximal Mesna activity for facilitating tissue separation when used in surgery. This maximal Mesna activity can be explained by the high sensitivity of a liquid solution of Mesna, for example of a liquid solution of Mesna in water, to oxidation. Dissolving Mesna in powder form shortly before use thus minimizes Mesna oxidation and as a result maximizes Mesna activity and alleviates the need for chelating and antibacterial agents. Maximization of Mesna activity is advantageous for use of Mesna in a method of surgery. The measure of preparing the composition by dissolving Mesna in powder form in one or more pharmaceutically acceptable solvents and/or one or more pharmaceutically acceptable buffers, at most one day prior to performing the method of surgery, also has the advantage that a composition comprising a tailor-made concentration of Mesna and volumes of the composition comprising Mesna can be prepared case-by-case in function of the needs for a method of surgery by dissolving a particular amount of Mesna in powder form in a particular amount of one or more pharmaceutically acceptable solvents and/or one or more pharmaceutically acceptable buffers. The use of tailor-made Mesna concentrations and volumes of compositions comprising Mesna is advantageous for facilitating tissue separation during surgery.

The invention further provides a kit for obtaining a composition comprising Mesna for use in a method of robot-assisted surgery.

A kit comprising Mesna in powder form next to one or more pharmaceutically acceptable solvents and/or one or more pharmaceutically acceptable buffers allows Mesna in powder form to be dissolved in the one or more pharmaceutically acceptable solvents and/or one or more pharmaceutically acceptable buffers shortly before using the resulting Mesna solution, thus minimizing the risk of Mesna oxidation and maximizing Mesna activity for facilitating tissue separation when used in surgery.

The invention further provides a robot instrument for chemically assisted mechanical dissection comprising:
a cartridge comprising a composition comprising Mesna in a liquid form;
a pump connectable, and preferably connected, to said cartridge in order to feed the composition comprising Mesna to an outlet tubing of said pump, upon actuation of said pump;
a robot arm comprising an irrigation channel with an inlet connectable, and preferably connected, to said outlet tubing of said pump and comprising an outlet connectable to an irrigation tip mountable, and preferably mounted on said robot arm, wherein said tip comprises at least one surgical tool selected from the group comprising scalpels, scissors, bovies, forceps, dissector, elevators, hooks, probes, needles, knot pushers, retractors, scopes, clamps and graspers and wherein the tip further comprises at least one irrigation orifice on said at least one surgical tool, said irrigation orifice fluidly connected to the irrigation channel;

whereby actuation of the pump supplies composition comprising Mesna from the cartridge through the irrigation channel to said irrigation tip of the mounted robot arm, and whereby the actuation of the pump is controllable by an operating switch.

The experience of using robots in surgery compared to other systems of open surgery or laparoscopic surgery show that the key factor in the success of the surgery is the skill and experience of the surgeon controlling the robot. A key aspect of the invention is to make possible the topical dispensing of a Mesna solution by the surgeon operating the mechanical dissection with the robot arms wherein the irrigation of Mesna is controlled by an operating switch, which is preferably a pedal under the foot of a surgeon.

Preferred embodiments of the robot instrument are provided. Within the robot instrument of the invention, an irrigation channel featuring an orifice for dispensing the Mesna solution is provided within the dissection tools at the tip of the robot arms for precise concomitant application of the dissection force and of the composition comprising Mesna.

The mechanical dissection tools can be of different types: spatulas, hooks, graspers, dissectors, etc. provided that they are suitable for applying a non-cutting mechanical separation force on the tissues.

The invention further provides a method of robot assisted surgery using composition comprising Mesna.

### BRIEF DESCRIPTION OF THE DRAWINGS

FIG. 1 shows a first embodiment of the device according to the invention.

FIGS. 2A to 2D show the steps of use of the device presented in FIG. 1.

FIG. 2A shows a cross section view of the device presented in FIG. 1 wherein a step of use of the device is shown, wherein in particular a movement of a trigger 3 to an up position is shown by an arrow.

FIG. 2B shows a cross section view of the device presented in FIG. 1 wherein a step of use of the device is shown, wherein in particular a pushing motion of an outlet tube 1′ towards a distal end Y of the device is shown by an arrow.

FIG. 2C shows a cross section view of the device presented in FIG. 1 wherein a step of use of the device is shown, wherein in particular a downward movement of a trigger 3 from its up position to its down position is shown by a continued arrow.

FIG. 2D shows a cross section view of the device presented in FIG. 1 wherein a step of use of the device is shown, wherein in particular an upward movement of a trigger 3 back to its up position is shown by an arrow, which upward movement is forced by a spring whenever not any force is applied on the trigger 3 in order to move it from the up position to the down position.

FIGS. 3A to 3C show the steps for obtaining the chambers of the device presented in FIG. 1.

FIG. 3A shows a tube 18 which membrane is made of a laminate material, of which laminate material further details are shown in the enlarged view.

FIG. 3B shows a tube 18 of FIG. 3E which is permanently sealed at one end 16.

FIG. 3C shows a tube 18 of FIG. 3F with a second seal 14 at a distance d from the permanent seal 16.

FIG. 4 shows a second embodiment of the device according to the invention.

FIGS. 5A to 5D show the steps of use of the device presented in FIG. 4.

FIG. 5A shows a cross section view of the device presented in FIG. 4 wherein a step of use of the device is shown, wherein in particular a movement of a trigger 3′ from an off position towards a handle 24 of the device is shown by an arrow.

FIG. 5B shows a cross section view of the device presented in FIG. 4 wherein a step of use of the device is shown, wherein in particular an arrow shows a movement of a linear ratchet 22 that forces a piercing means 20 to pierce and disrupt a disruptable membrane 26 of a first chamber 8.

FIG. 5C shows a cross section view of the device presented in FIG. 4 wherein a step of use of the device is shown, wherein in particular a pushing motion of an outlet tube 1′ towards a distal end Y of the device is shown by an arrow.

FIG. 5D shows a cross section view of the device presented in FIG. 4 wherein a step of use of the device is shown, wherein in particular an arrow indicates a pushing and/or squeezing motion of a trigger 3′ towards a handle 24 of the device.

FIG. 6A shows a perspective view of a device according to a third embodiment of the invention.

FIG. 6B shows a cross section view of the device of FIG. 6A wherein the chambers are not pierced.

FIG. 6C shows a cross section view of the device of FIG. 6A wherein the chambers are pierced.

FIG. 6D shows a closer cross section view of the pressure means and the outlet of the device of FIG. 6A.

FIG. 7A shows a cross section view of a device according to a fourth embodiment of the invention. Said device is closed position.

FIG. 7B shows a cross section view of the device shown in FIG. 7A wherein a second device, here a syringe, is connected to the device.

FIG. 7C shows a cross section view of the device shown in FIG. 7A after disconnecting the second device and in an intermediate position.

FIG. 8A shows a perspective view of a device according to a fifth embodiment of the invention wherein the air inlet is in closed position.

FIG. 8B shows a cross section view of the device shown in FIG. 8A.

FIG. 8C shows a closer cross section view of the device of FIG. 8A wherein the air inlet is in closed position.

FIG. 8D shows a closer cross section view of the device of FIG. 8A wherein the air inlet is in open position.

FIG. 8E shows a perspective view of the use of the device shown in FIG. 8A.

FIGS. 9A to 9G show the different steps of an embodiment of the use of a mucolytic agent in hydrosurgery.

FIG. 9A shows a disrupting device enclosed in a blister pack 250, which disrupting device is provided with a piercing means which is covered by a removable foil 251.

FIG. 9B shows a first container 252 containing a solute 253 initially sealed by a disruptable membrane, which disruptable membrane is pierced by pushing the piercing means of the disrupting device of FIG. 9A through the membrane, as indicated by arrows in FIG. 9B.

FIG. 9C shows an action of connecting, which action is indicated by arrows, of a first container 252 to a second container 254 using a connection means 256 such as a luer port which is fixed to the second container 254.

FIG. 9D shows an at least partial transfer of solvent from a second container 254 in to the first container 252 thereby dissolving the solute in a transferred solvent volume.

FIG. 9E shows an action of turning an assembly according to FIG. 9D, of which the direction of turning is indicated by arrows in FIG. 9E.

FIG. 9F shows transferring back a transferred solute volume in which a solute is dissolved into a second container 254 containing a remaining volume of the solvent.

FIG. 9G shows an action of disconnecting a first container 252 from a second container 254, of which the action of disconnecting is indicated by arrows in FIG. 9G.

FIG. 10 shows an embodiment of the system wherein a mucolytic agent is used in hydrosurgery.

FIG. 11 shows a perspective view of the robot instrument for chemically assisted mechanical dissection, comprised of a cartridge comprising a composition comprising Mesna in a liquid form, a peristaltic pump, a robot arm comprising an irrigation channel with an inlet connected, to said outlet tubing of said pump and a console whereby an operator surgeon is seated, and whereby the irrigation tip is controllable by an operating pedal.

FIG. 12 shows a perspective view of the robot arm comprising an irrigation tip mounted on said robot arm.

FIG. 13 shows a perspective view of the irrigation tip mounted on the robot arm.

FIG. 14 shows a cross-section in the longitudinal direction of the irrigation tip mounted on the robot arm, whereby said irrigation tip comprises a Maryland dissector and wherein the tip mounted on the robot arm further comprises one irrigation orifice within said Maryland dissector.

FIG. 15 shows a cross-section in the longitudinal direction of the irrigation tip mounted on the robot arm.

### DETAILED DESCRIPTION

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

As used herein, the following terms have the following meanings:

“A”, “an”, and “the” as used herein refers to both singular and plural referents unless the context clearly dictates otherwise. By way of example, “a compartment” refers to one or more than one compartment.

“About” as used herein referring to a measurable value such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed.

“Comprise,” “comprising,” and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

The recitation of numerical ranges by endpoints includes all numbers and fractions subsumed within that range, as well as the recited endpoints.

The expression “% by weight” (weight percent), here and throughout the description unless otherwise defined, refers to the relative weight of the respective component based on the overall weight of the formulation.

The term “dyes”, as disclosed herein, refers to substances which impart color to a substrate by selective absorption of light. Dyes are soluble and/or go through an application process which, at least temporarily, destroys any crystal structure of the color substances. Dyes are retained in the substrate by absorption, solution, and mechanical retention, or by ionic or covalent chemical bonds. The term “dyes” includes fluorescent and non-fluorescent dyes, which fluorescent dyes include without limitations fluorophores and which non-fluorescent agents include without limitations pigments, chemiluminescent compounds, luminescent compounds and chromophores.

The term “fluorophore” as used herein refers to a compound that is inherently fluorescent or demonstrates a change in fluorescence upon binding to a biological compound or metal ion, i.e., fluorogenic. Non-limiting examples of fluorophores are fluorescent proteins, inorganic fluorescent pigments and organic fluorescent dyes. Numerous fluorescent dyes are known to those skilled in the art and are described herein, and include, but are not limited to, indocyanine green, riboflavin, riboflavin 5′-phosphate sodium, methylene blue, semiconductor nanocrystals, coumarin, acridine, furan, indole, quinoline, cyanine, benzofuran, quinazolinone, benzazole, borapolyazaindacene and xanthenes, with the latter including fluoroscein, rhodamine, rhodol, rosamine and derivatives thereof. Preferably, the dyes are selected from the group consisting of indocyanine green, riboflavin, riboflavin 5′-phosphate sodium, methylene blue and fluorescein.

The term “pharmaceutically acceptable solvent”, as disclosed herein, refers to a solvent capable of dissolving relevant quantities of Mesna, and preferably capable of dissolving relevant quantities of Mesna in powder form, and preferably also capable of dissolving one or more dyes. Non-limiting examples of pharmaceutically acceptable solvents are water, physiological serum, and water solution comprising up to 0.9 mass/volume percent sodium chloride. Bases, such as sodium hydroxide, may be added to the pharmaceutically acceptable solvent. Preferable, said pharmaceutically acceptable solvent is sterile. Further examples of pharmaceutically acceptable solvents are known to the person skilled in the art.

The term “pharmaceutically acceptable buffer”, as disclosed herein, refers to a solution comprising at least one buffer excipient, capable of keeping pH of a composition comprising Mesna, and also of a composition comprising Mesna and one or more dyes, at a nearly constant value. The buffer might be obtained by mixing a solvent with at least one buffer excipient. The solvent might be water, might consist of water of high purity (e.g. water for injection as per pharmaceutical standard) and may contain between 0 and 0.9 mass/volume percent of NaCl. The presence of NaCl reduces the side-effects due to chlorine ion depletion. In particular, lack of sufficient chlorine ions may trigger unexpected hyperactivity of some cells such as neurons in brain and in the nerves. Pharmaceutically acceptable buffers are known to the person skilled in the art. According to preferred embodiments, said buffer excipient is selected from the group comprising sodium acetate, acetic acid, glacial acetic acid, ammonium acetate, arginine, aspartic acid, benzoate sodium, benzoate acid, carbonate sodium, bicarbonate sodium, citrate acid, citrate sodium, citrate disodium, citrate trisodium, glucono delta lactone, glycine, glycine HCl, histidine, histidine HCl, hydrobromic acid, meglumine, phosphate acid, phosphate monobasic potassium, phosphate dibasic potassium, phosphate monobasic sodium, phosphate dibasic sodium (Na₂HPO₄), tartrate sodium, tartrate acid, tromethamine (tris) or any combination thereof. In preferred embodiments, the concentration of the buffer excipient is of from 2 mM to 60 mM, preferably from 2.5 mM to 50 mM, more preferably from 5 to 40 mM, more preferably from 7.5 mM to 35 mM, most preferably from 10 mM to 30 mM. According to a preferred embodiment, the pH of the buffer may be adjusted using pH adjustment agents to reach a desired pH of at least 8.5. Said pH of the buffer allows obtaining a composition comprising Mesna and one or more dyes having a pH of from 6 to 8 by only adding solid Mesna in powder form or in lyophilized form, and one or more dyes, to the buffer and without addition of pH adjustment agents. This avoids manipulation of the obtained composition comprising Mesna and one or more dyes, to adjust the pH for instance, thereby providing for the immediate use of the obtained composition. This ensures a maximal sterility of the composition and a maximal safety for a patient thanks to the use of a solution with a pH close to physiological pH. According to a preferred embodiment, the buffer comprises phosphate such that the phosphate to Mesna ratio of the composition comprising Mesna is at least 1/500, preferably at least 1/400, more preferably at least 1/300, even more preferably at least 1/250, most preferably at least 1/150, even most preferably at least 1/100. Said ratio is at most 1/1, preferably at most 1/1.5, more preferably at most 1/2, most preferably 1/3.

In the present text, “Mesna” is used as an abbreviation for sodium-2-mercaptoethane sulfonate. Mesna is a white hygroscopic powder with a characteristic odor. The liquid solution in water is highly sensitive to oxidation and rapidly decomposes on contact with oxygen to form di-Mesna also called disulphide Mesna, which is the inactive degradation product of Mesna, and which di-Mesna is poorly absorbed, particularly in a humid atmosphere. Mesna is known to break molecular bonds between tissue layers, thereby facilitating tissue separation. Specifically, Mesna breaks disulfide bonds of polypeptide chains and proteins, which disulfide bonds are responsible for the adherence of pathological tissues and for the strength of fibrosis.

The term “chemically assisted tissue dissection”, as disclosed herein, refers to a method of dissection using Mesna as auxiliary chemical substance for use in surgery.

The term “chemically assisted tissue dissection systems”, as disclosed herein, refers to systems indicated for the cleavage and separation of tissue layers to facilitate the detachment of tissues and the dissection of fibrosis in various surgical procedures for patients. In particular, the systems are suitable for use in open surgery as well as in non-invasive surgery (endoscopic and laparoscopic surgery). Preferably, chemically assisted tissue dissection systems are selected that are classified as Class III devices per Rule 13 of Annex IX to Medical Device Directive 93/42/EEC, as amended, or as combination devices by the US FDA.

The term “robotic assisted surgery”, as disclosed herein, is to be understood as a surgical technology that places a computer-assisted electromechanical device, also to be construed as a robot or a robotic device, in the path between a medical practitioner performing the surgery and a patient being operated. Robotic-assisted surgery can be applied in open surgery as well as in non-invasive surgery, with endoscopic and laparoscopic surgery as examples of non-invasive surgery. Types of applications wherein robotic-assisted surgery can be applied include skull base surgery, open surgery, non-invasive surgery, thoracic surgery, cardiovascular surgery, dentistry, orthopedic surgery, plastic surgery, ophthalmic surgery, neck surgery, neurosurgery, maxillo-facial surgery, gallbladder surgery, endoscopic surgery laparoscopic surgery, vascular surgery, colorectal surgery, general surgery, gynecologic surgery, heart surgery, endometriosis, head and neck surgery, transoral head and neck surgery, urologic surgery. Robotic assisted surgery shows many benefits. Robotic assisted surgery benefits a patient directly, in terms of a shorter recovery time, as well as indirectly, in terms of a better visualization available to a medical practitioner performing the surgery, leading to a more precise surgery. Other benefits include a greater range of motion and dexterity for a medical practitioner performing the surgery, the availability of a highly-magnified, high-resolution image of an operating field related to a surgical procedure, better access to body parts being operated, a shorter hospital stay, less risk of infection, less blood loss and fewer blood transfusions, less pain, faster recovery and a quicker return of a patient to daily routine. Preferably, said computer-assisted electromechanical device, for which “robotic surgical system” is used as a synonym in the present text, comprises at least one steerable robotic arm. Said at least one steerable robotic arm may be directly or indirectly supportable, and preferably supported, by a cart. Preferably, such robotic surgical system further comprises a console from which the at least one robotic arm can be controlled by providing instructions to the console. The at least one arm is configured to hold objects and/or comprises one or more terminally located surgical tools. In case of laparoscopy, such tools may be construed as laparoscopic instruments. Non-limiting examples of said tools are cannulas, scalpels, scissors, bovies, forceps, dissector, elevators, hooks, probes, needles, knot pushers, retractors, scopes and graspers. The term “dissector”, as used herein, can be understood as forceps which are configured to operate interchangeably in a grasping mode, i.e. for grasping and exerting a pulling force on a tissue and in a dissection mode, i.e. for dissecting tissues. According to a preferred embodiment, a dissector comprises two opposite jaws which can be closed into tissue to exert a pulling force (grasping mode), and, when the jaws are opened, which dissector is configured to mechanically separate tissues by lateral forces (dissection mode).

The term “laparoscopy” or “laparoscopic surgery”, as disclosed herein, refers to minimally invasive abdomen surgery performed with the aid of an optical system. The term “laparoscopic instruments”, as used in the present text, is to be understood as one or more instruments suitable for enabling laparoscopy performed on a patient.

The present invention provides a device for delivering a solution comprising at least one solvent and at least one solute. Preferably, the solution consists of one solute and one solvent. Said solute is a mucolytic agent in powder form. In a preferred embodiment, said mucolytic agent is sodium 2-mercaptoethanesulfonate known as Mesna. In a preferred embodiment, Mesna is used in a powder form. In a preferred embodiment, said solute is saline water wherein the NaCl content is adjusted to make the solution isotonic. The solution, also called herein Mesna solution, the mixture, the Mesna mixture or the solution is shortly prepared prior use.

Preferably, the device comprises at least one control means which provides for the control of the solution volume delivered and flowing out of the device.

In a preferred embodiment, the device comprises a first chamber for housing the solute, a second chamber for housing the solvent and at least one outlet suitable to be in fluid communication with at least one of the chambers; said chambers are separated from each other by at least one disruptable separation means and are in fluid communication with each other upon disruption of said separation means.

Preferably, the separation means comprise a spatial separation between the first chamber and the second chamber. This means that when the device is not in use, the chambers do not share any common element, such as membranes and/or walls. In this embodiment, when the device is used, at least one of said chambers is movable towards the other chamber and/or both chambers are movable towards each other. One of the advantages of this design is that it offers the possibility to fill the chambers separately from each other in a sterile environment. Assembling the device can then later be performed in a non-sterile environment. After assembling the device, an extra sterilization step can be performed.

The first chamber and the second chamber can be also attached to each other by said separation means. This means that when the device is in use, it is not required to move at least one of the chambers towards the other chamber. Said chambers are fluidly connected to each other upon removal of the separation means.

Preferably, the device comprises at least one disruption means for disrupting said separation means. Preferably, the device further comprises a disruptable sealing membrane positioned between the outlet and the chamber which is suitable to be in fluid communication with said outlet.

In a preferred embodiment, the device comprises a single chamber for housing the solute, at least one connection means for fluidly connecting said device to an external solvent source and at least one outlet suitable to be in fluid communication with said single chamber. Preferably, said solute is a mucolytic agent in powder form. This embodiment provides a device which is easy to use, allows delivering high Mesna solution volumes and can be used in any operating room surgery. The device further presents a very low production cost.

Preferably, the connection means connects said device, more in particular the single chamber of the device, to a solvent source. The connection means can be any means known to the person skilled in the art. Said means can be an opening or a tube which is connectable to a solvent source or line or the outlet of the device itself. The device preferably comprises a plunger, or any similar system known to the person skilled in the art, for the suction of the solvent from the solvent source into the single chamber of the device through the connection means.

The solvent volume which will be inserted into the single chamber of the device can be a predefined amount that is fully inserted in said chamber. The practitioner can hence select the concentration of the Mesna solution that is deemed required for the target location, for the type of the tissue to be separated and/or the type of the intervention. Said concentration will of course depend on the amount of Mesna powder provided in the single chamber of the device and the solvent volume introduced by the practitioner into said chamber. In a preferred embodiment, the device is provided with an information sheet comprising at least an indication of the Mesna powder amount contained in the single chamber of the device.

After obtaining the Mesna solution, at the desired concentration, inside the single chamber of the device, the connection between the single chamber and the solvent source can be removed so as to prevent insertion of more solvent into said chamber. The solution can then be delivered to the target.

In a preferred embodiment, the device comprises a single chamber for housing the solute suitable to be connected to a solvent reservoir in order to prepare a solution. Said reservoir is connectable to a solvent jet surgery system known to the person skilled in the art as water jet surgery system. Said reservoir replaces the physiological water bag present in conventional known water jet surgery systems. The single chamber is provided with the required fluid connection for connecting it to a solvent reservoir. Said connection is known to the person skilled in the art. The invention allows delivering Mesna solution contained in said reservoir at high pressure for assisting surgery. A multifunction device is thereby achieved, which combines the advantages of water jet surgery, including injection of the liquid and/or dissection by means of liquid, with the advantages of the delivered Mesna solution. In particular, dissection is made possible at lower jet pressures, minimizing the risks of accidental damage to the surrounding tissues and extending the range of possible applications of water jet surgery. Hence, the device according to the invention makes it possible to inject liquid and/or the Mesna solution, obtained in the single chamber, to the target location with high pressure thereby lifting the target away from other surrounding tissues. Device for water jet surgery are known to the person skilled in the art and are for instance disclosed in U.S. Pat. No. 5,116,313 and US 2009/0069805.

After obtaining the Mesna solution, at the desired concentration, inside said reservoir, the device is connected to the water jet surgery system. Delivery of Mesna solution under pressure to the target location can be initiated. In a preferred embodiment, the device, more in particular the single chamber containing Mesna solution, replaces the physiological water bag present in conventional known water jet surgery systems. The water jet surgery system preferably comprises a delivery means selected from the group comprising: a surgical device, a high pressure pump, a delivery tube or applicator or any combination thereof. The Mesna solution is delivered via said delivery means to the target location. The surgical device is as described above.

The present invention further provides a method for weakening inter-tissues and/or organs adhesion by delivering a solution comprising at least one solvent and at least one solute to tissues and/or organs. Preferably, the solution consists of one solute and one solvent. The method comprises the steps of dissolving said solute in said solvent thereby obtaining the solution, and delivering the obtained solution to said inter-tissues and/or organs. Preferably, the solute is a mucolytic agent in powder form. Preferably said solvent is a sterile physiological saline solution.

In a preferred embodiment, the method further comprises the steps of providing a device comprising two chambers as described above, disrupting the separation means of the device thereby obtaining the solution, and delivering the obtained solution through the outlet of the device to a target location.

In another embodiment, the method comprises the steps of providing a device comprising a single chamber as described above, connecting said device and more in particular said single chamber to an external solvent source, inserting the solvent into said single chamber thereby obtaining the solution, i.e. Mesna solution, and delivering the obtained solution through the outlet of the device to a target location.

In another embodiment, the method comprises the steps of providing a device comprising a single chamber as described above, inserting the solvent into said single chamber thereby obtaining the solution, i.e. Mesna solution, connecting said device and more in particular said single chamber to a water jet surgery system, and delivering the obtained solution to a target location. Preferably, the solution consists of one solute and one solvent. Said delivery is made through a delivery means of water jet surgery system thereby, delivering Mesna solution to the target location at high pressure. Said delivery means is selected from the group comprising: a surgical device, a high pressure pump, a delivery tube, an applicator or any combination thereof.

The method according to any embodiment of the invention provides for the controlled delivery of Mesna solution. Said controlled delivery might be performed using control means of the device and/or using an electrically driven mechanical system. Said system can be a syringe driver, a syringe pump or any other system known to the person skilled in the art. Said system is preferably controlled by the practitioner using a pedal connected to the electrically driven mechanical system. This is advantageous as it provides the practitioner with a high hands freedom level required for instance for laparoscopic surgery also called minimal invasive surgery. Indeed, said practitioner will be able to use both hands for operations other than handling the device for delivering Mesna solution to the target location.

It is to be understood that for all the embodiments of the device and/or the method of the present invention, the solute amount contained in the device and/or the solvent volume are selected such as the solute concentration in the solution is of from 5% to 50%, preferably of from 10% to 40%, more preferably from 15% to 30%, most preferably from 20% to 25% or any value comprised in between the mentioned ranges.

In a preferred embodiment, the solvent is saline water wherein the content of NaCl is adjusted to make the solution isotonic.

The device according to any embodiment of the present invention comprises at least one sterilization microfilter positioned between the outlet of the device and the chamber which is suitable to be in fluid communication with said outlet. Preferably said microfilter is a membrane made of polyethylene terephtalate, polyamide, polyethersulfone, nylon or any other suitable material. In a preferred embodiment, the pore size is from 0.1 to 3 μm, preferably from 0.15 to 2 μm, more preferably from 0.2 to 1 μm, most preferably about 0.22 μm. The presence of said microfilter further enhances the sterility of the solution and/or ensures the instillation of the sterile solution of the device, i.e. Mesna solution, during dissection. In a preferred embodiment, said microfilter has an additional ventilation membrane to remove air bubbles during dispensing and letting air in the chamber to compensate for liquid volume dispensed. In a preferred embodiment, the pore size of the ventilation membrane is from 0.01 to 0.05 μm, preferably about 0.02 μm. The ventilation membrane is preferably made of polytetrafluoroethylene or any other suitable material.

It is to be understood that for all the embodiments of the device and/or the method of the present invention, the Mesna in powder contained in the device is sterilized, preferably by gamma irradiation or X-ray. The irradiation is preferably performed at least at 20 kGy, preferably at least 25 kGy, more preferably at least 30 kGy and at most 50 kGy, preferably at most 40 kGy.

The outlet of the device, according to any embodiment of the present invention, is suitable to be connected to a surgical device or to a delivery tube. Said outlet might be connected via a luer lock mechanism to said surgical device. The surgical device can be a dissector instrument, known to the person skilled in the art, which primary function is tissues separation in surgery through mechanical action. Said dissector is a general manual surgical instrument used in gynecology, ENT surgery, orthopedics, neurosurgical and all other surgical procedures where tissues need to be separated. Said dissectors are stainless steel or titanium instruments with different hooks and curves, such as long-medium, short beveled hook, hook with ball, straight and curved, big and small size versions. The dissectors may be fitted with internal capillary tube for dispensing the fluid at their active edge. The shape of the dissector to be used in surgery depends on the application and area. Many dissectors are re-usable or disposable, in plastic, stainless steel, titanium or other metals. The dissector can also be fitted with a cavity and a second capillary tube for the suction of the excess liquids and Mesna solution, as commonly used for removing excess liquids from the operating field during the procedure that would otherwise prevent complete vision of the field. In a preferred embodiment, said surgical device or delivery tube can be combined with suction and/or suction/irrigation devices used in open and minimally invasive surgery. For minimally invasive surgery, said surgical device or delivery tube can be inserted in the instrument channel of suction/irrigation devices.

The device, according to any embodiment of the present invention, is connectable to and/or controllable by an electrically driven mechanical system for the delivery of Mesna solution to the target location. Said system can be a syringe driver, a syringe pump or any other system known to the person skilled in the art. Said system is preferably controlled by the practitioner using a pedal connected to the electrically driven mechanical system. This is advantageous as it provides the practitioner with a high hands freedom level required for instance for laparoscopic surgery also called minimal invasive surgery. Indeed, said practitioner will be able to use both hands for operations other than handling the device for delivering Mesna solution to the target location.

The device, according to any embodiment of the present invention can also be hand held and hand manipulated by the operator. Where required, the device is further provided with a plunger which is used by the operator for delivering said Mesna solution. Said plunger is slidably engaged into one chamber of the device.

The device, according to any embodiment of the invention, is a single-use device. By preference, it is a single-use chemically assisted tissue dissector indicated for the cleavage and separation of tissue layers to facilitate various surgical procedures, including abdominal surgery, gynecology, orthopedics and otoneurosurgery.

The device and/or the method according to any embodiment of the present invention, allows mixing Mesna powder with the solvent shortly prior use thereby making it possible to provide a Mesna solution having a maximum Mesna activity as oxidation of said Mesna is reduced compared to Mesna solutions stored for a long time prior use. Said Mesna solution is prepared and/or delivered to the target at most 24 hours, preferably at most 12 hours, more preferably at most 6 hours, even more preferably at most 4 hours, most preferably at most 30 min, even most preferably at most 10 min after dissolving the solute in the solvent. In a further preferred embodiment, said solution is delivered at most 5 min, preferably at most 4 min, more preferably at most 3 min, most preferably immediately, i.e. 0 min, after dissolving the solute in the solvent. In a preferred embodiment, the solvent is suitable for dissolving the mucolytic agent in powder form. Preferably said solvent is a sterile physiological saline solution. In a preferred embodiment, the device is designed to contain 5 to 60 ml, preferably 10 to 50 ml, more preferably 12 to 40 ml, most preferably 15 to 30 ml of solvent or any volume comprised within the mentioned ranges. Preferably, the device is designed to contain about 20 ml of solvent.

The present invention further provides a kit comprising Mesna in powder form, a solvent for dissolving said Mesna and a device according to any embodiment of the invention. In a preferred embodiment, the device comprises said Mesna in powder form and/or said solvent. In a preferred embodiment, the solvent is saline water wherein the content of NaCl is adjusted to make the solution isotonic. The kit further comprises a leaflet provided with user's instructions and/or information on the Mesna and/or the solvent and/or the device of said kit.

The different embodiments of the device will now be described with reference to the accompanying figures.

Referring to FIG. 1, a first embodiment of the device is shown. The device comprises a first chamber 8 housing a first substance and a second chamber 6 housing a second substance. In FIG. 1, the first chamber comprises the first substance in powder form, i.e. the solute and the second chamber comprises the liquid in which said first substance will be dissolved, i.e. the solvent. In a preferred embodiment, said first substance in powder form is Mesna. The chambers are separated from each other by a disruptable separation means. In this embodiment, the chambers are also attached to each other by the same disruptable separation means which is in this case a disruptable membrane 7. The first chamber 8 is sealed by a disruptable sealing membrane 2. The proximal end X of the device is provided with an outlet 1 and an outlet tube 1′ for guiding the mixture out of the device. The outlet tube 1′ is movable and its distal end Y is suitable to disrupt the sealing membrane 2 of the first chamber 8. Said distal end of the outlet tube 1′ might have a pointed shape as shown in FIG. 1 or might be of any other type and/or shape suitable to disrupt the sealing membrane 2. The device further comprises a microfilter 9 positioned between the outlet 1 of the device and the first chamber 8 which is suitable to be in fluid communication with said outlet 1. Preferably, the microfilter is integrated with and/or within the outlet tube 1′. Said microfilter enhances the sterility and prevents contamination of the device and the solution and/or powder contained therein. In a preferred embodiment, said microfilter is made of polyethylene terephtalate, nylon, polyethersulfone or polyamide membrane.

The device shown in FIG. 1 is further provided with a disruption means for disrupting the separation means, i.e. disruptable membrane 7. Said disruption means comprises a lever 4, a trigger 3 and a gear ratchet 5, preferably a gear with locking ratchet. The trigger 3 is movable from a position in which its proximal end X is in contact with the device, called down position, to a position in which the proximal end X of the trigger 3 is not in contact with the device, called up position. The lever 4 is in contact with the gear ratchet 5 and also in contact with one of the chambers, preferably with the chamber 6 which is at the distal end Y of the device, more preferably with the device containing the liquid solvent. With this design, the movement of the trigger 3 leads to the rotation of the gear ratchet 5 which in turn moves the lever 4, thereby applying a pressure on the chamber 6.

In use, the user moves the trigger 3 to the up position as shown in FIG. 2A. Said trigger 3 might be fixed to the device and/or to the lever 4 by a snap fit system, or any other removable fixation means known to the person skilled in the art, when the device is not in use (FIG. 1). Then, the user moves the trigger 3 from its up position towards its down position as shown by the arrow in FIG. 2A. By doing so, the trigger 3 and/or gear ratchet 5 move the lever 4 in the same direction as the movement of the trigger 3, thereby applying a pressure on the liquid chamber 6. Said movement and/or pressure leads to the disruption of the separation means, i.e. disruptable membrane 7 thereby fluidly connecting both chambers, as shown by element 17. The content of both chambers 6 and 8 are brought together thereby dissolving the solute in the solvent and obtaining the solution as shown in FIG. 2A. The device can also be designed such as the disruption of the separation means caused by a pressure applied by the user on the lever 4 of the device (as shown by the arrow in FIG. 2A).

Afterwards, the user pushes the outlet tube 1′ towards the distal end Y of the device as shown by the arrow in FIG. 2B. The outlet tube 1′ might be pushed by connecting the outlet 1 of the device to any other device such as a surgical tissue dissector. Said outlet tube might also be twisted by connecting the outlet 1 of the device to any other device such as a surgical tissue dissector. Said outlet tube 1′ will disrupt the sealing membrane 2 and will be in fluid contact with the fluidly connected chambers 17 of the device FIG. 2B. In the next step, the user moves the trigger 3 from its up position towards its down position as shown by the continued arrow of FIG. 2C. The lever 4 will then be moved in the same direction as the movement of the trigger 3, thereby applying a pressure on the liquid chamber 6 leading to the flow of the solution out of the device through the outlet tube and the outlet 1 of the device as shown by the discontinued arrow in FIG. 2C. The gear ratchet 5 might be provided with a spring which is extended when the trigger 3 moves towards its down position. Said spring forces the trigger 3 to go back to its up position (shown in FIG. 2D) whenever the uses not applying any force on the trigger 3 in order to move it from the up position to the down position.

The device is simple to use and offers a rapid system for dissolving the solute in the solvent. The device is also practical as it is suitable to be manually held by the user such as said holding is comparable to a pen holding wherein the index finger is moving the trigger 3 of the device.

The device is provided with control means for the control of fluid volume flowing out of the device. Said control means comprises the trigger 3, the gear ratchet 5 and the lever 4 which are designed and/or positioned such as a predetermined volume of solution is flowing out of the device with each movement of the trigger 3 from its up position to its down position. In this way, the user can have a control over the amount of fluid flowing out of the device, thereby avoiding any excess of delivered solution volume. In addition, the user will be provided with a control over the time at which the fluid is flowing out of the device. These possibilities are not offered by the devices of the prior art.

FIG. 3 shows the steps for obtaining the chambers of the device presented in FIG. 1. A sleeve or a tube 18 (FIG. 3A) is permanently sealed at one end 16. A second seal 14 is then created at a distance d from the permanent seal 16 thereby creating a chamber. The second created seal 14 is a frangible seal and corresponds to the separation means, i.e. disruptable membrane 7 of the device shown in FIG. 1. The distance d is dependent on the diameter of the tube 18 and/or on the solvent's volume or the amount of powder that will be introduced in the created chamber. In a preferred embodiment, the permanent seal 16 and the frangible seal 14 are created by heat sealing the tube 18 at desired positions.

In a preferred embodiment, the sleeve or tube membrane is made of a laminate material as shown in the enlarged view of FIG. 3A. Said laminate material comprises at least an adhesive layer 12 having two surfaces, wherein one surface is covered by a layer 10 of high heat seal resistant material and the other surface is covered by a layer 13 which is preferably made from polypropylene or polyethylene or a combination thereof. In a preferred embodiment, the high heat seal resistant material layer 10 is the outer surface of the tube 18 and the layer 13 made from polypropylene or polyethylene or a combination thereof is the inner surface of the tube 18 (FIG. 3A). In a preferred embodiment, the laminate material is a polyethylene laminate, more preferably, polychlorotrifluorethylene laminate.

In a preferred embodiment, the trigger and/or the lever and/or the gear with locking ratchet and/or the outer walls of the device are made of injectable plastic material. In a preferred embodiment, the separation means and/or the sealing membrane are made of aluminium laminate.

Referring to FIG. 4, a second embodiment of the device is shown. The device comprises a first chamber 8 housing a first substance and a second chamber 6 housing a second substance. In FIG. 4, the first chamber comprises the first substance in powder form, i.e. the solute and the second chamber comprises the liquid in which said first substance will be dissolved, i.e. the solvent. In a preferred embodiment, said first substance in powder form is Mesna. At least one of the walls of each chamber is at least partially made of a disruptable membrane. The chambers are separated from each other by a separation means which comprises a spatial separation between the first chamber and the second chamber. This means that the chambers are spatially separated from each other. The separation means further comprises the disruptable membranes 26, 27 of said chambers. In this embodiment, at least one of said chambers is movable towards the other chamber which can be movable or non-movable. Preferably said movement is a sliding movement. The first chamber 8 is sealed by a disruptable sealing membrane 2 which is not forming a part of the separation means. The proximal end X of the device is provided with an outlet 1 and an outlet tube 1′ for guiding the mixture out of the device. The outlet tube 1′ is movable and its distal end Y is suitable to disrupt the sealing membrane 2 of the first chamber 8. Said distal end of the outlet tube 1′ might have a pointed shape as shown in FIG. 4 or might be of any other type and/or shape suitable to disrupt the sealing membrane 2. The device further comprises a microfilter 9 positioned between the outlet 1 of the device and the first chamber 8 which is suitable to be in fluid communication with said outlet 1. Preferably, the microfilter is integrated with and/or within the outlet tube 1′. Said microfilter enhances the sterility and prevents contamination of the device and the solution and/or powder contained therein. In a preferred embodiment, said microfilter is made of polyethylene terephtalate, polyethersulfone, nylon or polyamide membrane. The device is also provided with a handle 24 via which the device is hold in a way similar to a pistol hold.

The device shown in FIG. 4 is further provided with a disruption means for disrupting the separation means. Said disruption means comprises a piercing means 20, trigger 3′, a linear ratchet 22 and a plunger 21. The piercing means is positioned between the first chamber 8 and the second chamber 6; preferably said piercing means 20 is positioned between the disruptable membranes 26, 27 of the chambers which are part of the separation means of the device. Said piercing means 20 is provided with at least two opposed piercing members 30, 31 for piercing and disrupting the disruptable membranes 26, 27 of the first and the second chambers. The piercing means 20 might be fixed to the first chamber 8 as shown in FIG. 4. Alternatively, said piercing means 20 might be fixed to the second chamber 6 or to the walls of both chambers. The trigger 3′ is suitable to be squeezed or pushed towards the handle 24 of the device. Said trigger 3′ is connected to a sliding block 28 having a drive tooth 29 suitable to engage a tooth of the linear ratchet 22 (FIG. 4). When the device is not used, the trigger 3′ is in “off position” in which it is not pushed in the handle 24 and the drive tooth 29 is engaging the most proximal tooth of the linear ratchet 22 as shown in FIG. 4. Preferably, the shape of the proximal end X of the linear ratchet 22 is form fitting the plunger 21 distal end Y. Said plunger 21 is positioned between the linear ratchet 22 and one of the chambers of the device, preferably the second chamber 6 containing the solvent as shown in FIG. 4. The distal end Y of the plunger 21 might be provided with a disruptable sealing membrane 23 (FIG. 4).

In use, the user moves the trigger 3′ from its “off position” towards the handle 24 of the device as shown by the arrow in FIG. 5A. The movement of the trigger 3′ leads to the movement of the sliding block 28 towards the distal end Y of the device thereby leading to the movement, i.e. sliding, of the drive tooth 29 which will engage the neighboring teeth of the linear ratchet 22. By neighboring teeth we refer the teeth positioned at the distal end Y of the ratchet teeth that is engaged by the drive tooth 29. The linear ratchet 22 will then be moved towards the proximal end X of the device and will disrupt the disruptable sealing membrane 23 of the plunger 21. The movement of the linear ratchet 22 continues leading to the engagement of the proximal end X of said ratchet 22 in the distal end Y of the plunger 21 as shown in FIG. 5A. The movement of the linear ratchet 22 towards the proximal end X of the device continues thereby moving the second chamber 6 in the same direction and forcing said chamber to come in contact with the piercing member 20 which will pierce and disrupt the disruptable membrane 27 of the second chamber 6 which is part of the separation means of the device (FIG. 5B). The piercing means 20 is designed to pierce and disrupt the disruptable membrane of the second chamber 6 without being completely inserted inside said chamber 6 as shown in FIG. 5B. The disruption of the disruptable membrane 27 of the second chamber 6 is performed by the piercing member 30 which is in close vicinity of said disruptable membrane 27. The movement of the linear ratchet 22 continues thereby forcing the piercing means 20 to pierce and disrupt the disruptable membrane 26 of the first chamber 8 as shown in FIG. 5B. It is to be understood that the piercing and the disruption of the disruptable membranes 26, 27 of the chambers can performed subsequently or simultaneously. In the latter case, the second chamber 6 is moved towards the proximal end X of the device until the opposed piercing members 30, 31 of the piercing means 20 are in contact with the disruptable membranes 26, 27 of the chambers. By continuing the movement of the second chamber towards the proximal end X of the device, both disruptable members are simultaneously disrupted.

The disruption of the disruptable membrane 26 of the first chamber 8 is performed by the piercing member 31 which is in close vicinity of said disruptable membrane 26 and which is opposing the piercing member 30 used for disruption of the membrane of the second chamber 6.

After disruption of the disruptable membranes 26, 27, the chambers 8, 6 will be fluidly connected to each other 17. The respective content of said chambers will merge. The solute will dissolve in the solvent thereby obtaining a solution as shown in FIG. 5B. In a next step, the user pushes the outlet tube 1′ towards the distal end Y of the device as shown by the arrow in FIG. 5C. Said outlet tube 1′ will disrupt the sealing membrane 2 and will be in fluid contact with the fluidly connected chambers 17 of the device FIG. 5D. The outlet tube 1′ might be pushed and/or twisted by connecting the outlet 1 of the device to any other device such as a surgical tissue dissector. Afterwards, the user pushes and/or squeezes further the trigger 3′ towards the handle 24 of the device as shown by the arrow in FIG. 5D thereby leading to the flow of the solution out of the device through the outlet 1.

In a preferred embodiment, the device is provided with at least one air vent 25 for evacuating air from the device when the first chamber 8 is moved towards the second chamber 6. Said air vent 25 is preferably provided at the proximal end X of the device outer walls more preferably at the level of the second chamber or the piercing member 20 when starting from the distal end Y of the device towards its proximal end.

The device is simple to use and offers a rapid system for dissolving the solute in the solvent. The device is also practical as it is suitable to be manually held by the user such as said holding is comparable to a pistol holding wherein the index finger is moving the trigger 3′ of the device.

The device is provided with control means for the control of fluid volume flowing out of the device. Said control means comprises the trigger 3′, the sliding block 28 and the linear ratchet 22 which are designed and/or positioned such as a predetermined volume of solution is flowing out of the device with each movement of the drive tooth 29. By movement of the drive tooth, we refer to the sliding of said drive tooth by which the drive tooth 29 will engage the neighboring teeth of the linear ratchet 22. In this way, the user can have a control over the amount of fluid flowing out of the device, thereby avoiding any excess of delivered solution volume. In addition, the user will be provided with a control over the time at which the fluid is flowing out of the device. These possibilities are not offered by the devices of the prior art.

Referring to FIG. 6A, a third embodiment of the device is shown. The device comprises a first chamber 50 for housing the solute, i.e. Mesna in powder form; a second chamber 51 for housing the solvent and at least one outlet 55 for delivering the solution. Said outlet is suitable to be in fluid communication with at least one of the chambers. On FIG. 6A, the outlet 55 is suitable to be in fluid communication with the second chamber 51. Said outlet 55 is covered by a removable cap (not shown) when the device is not used.

The chambers are separated from each other by at least one separation means comprising at least one disruptable separation means (52, 53 in FIG. 6B) and are in fluid communication with each other upon disruption of said disruptable separation means. At least one of the walls of each chamber is at least partially made of a disruptable membrane thereby forming the disruptable separation means.

FIG. 6B shows the details of the separation means. The chambers are separated from each other by a separation means which comprises a spatial separation between the first chamber 50 and the second chamber 51. This means that the chambers are spatially separated from each other and when the device is not in use, the chambers do not share any common element, such as membranes and/or walls. The separation means further comprises the above mentioned disruptable membranes 52, 53 of said chambers. At least one of said chambers is movable towards the other chamber which can be movable or non-movable. Said movement can be a sliding and/or a rotating movement. By preference, the first chamber 50 is movable towards the second chamber 51 which is non-movable. The first chamber 50 is sealed by a non-disruptable sealing membrane 60 which is not forming a part of the separation means.

The device is further provided with a disruption means 56 for disrupting the disruptable separation means, more in particular for disrupting the disruptable membranes 52 and 53. Said disruption means comprises at least one piercing means 56. The piercing means is positioned between the first chamber 50 and the second chamber 51. Preferably said piercing means 56 is comprised in the separation means and is positioned between the disruptable membranes 52, 53 of the chambers which are part of the separation means of the device. Said piercing means 56 is provided with at least two opposed piercing members for piercing and disrupting the disruptable membranes 52, 53 of the first and the second chambers (FIG. 6B). In a preferred embodiment, the disruptable membranes 52, 53 are made of aluminium laminate.

In a preferred embodiment, the device is provided with at least one air vent 57 for evacuating and/or inserting air from the device. Said air vent is covered by non-disruptable sealing membrane 60 when the device is not used (FIG. 6B). Said air vent 57 is preferably provided at the distal end W of the device outer wall. FIG. 6C shows a cross section view of the device wherein the chambers are pierced by the piercing means 56. Details of the proximal end of the device are shown on FIG. 6D. The proximal end Z of the device is provided with an outlet 55 for expelling the Mesna solution out of the device. Said outlet 55 is covered by a removable cap (not shown) when the device is not used.

In use, the user moves the first chamber 50 towards the second chamber 51. The movement leads to the disruption of the disruptable membranes 52, 53 by the piercing means 56 (FIG. 6C). The content of the first and the second chambers will merge to obtain the Mesna solution in which the solute is dissolved in the solvent. The device can be further agitated or shacked to further mix the solvent and the solute. Afterwards, the removable cap is removed thereby uncovering the outlet 55. The foil tab 60 is then removed for allowing air to replace the volume of the dispensed solution. The outlet of the device can then be used for direct delivery of the Mesna solution to the target or can be connected to any other suitable device such as a dissector 61. It is to be understood that the air vent 57 and the tab 60 can be replaced by an air permeable membrane.

In a preferred embodiment, the device comprises at least one pressure means 58 for manually applying a pressure on the first chamber walls and/or the second chamber walls thereby delivering the solution to said tissues and/or organs. The pressure means 58 is preferably visible to the user. The pressure means 58 can be a flexible button. Preferably, the second chamber is fitted with the pressure means 58. Said pressure means 58 is suitable for the application of a manual pressure thereby delivering the solution to said tissues and/or organs. The uncovered air vent 57 admits air when the flexible wall of the chamber returns to its stable position.

In a preferred embodiment, the invention provides a method for weakening inter-tissues and/or organs adhesion by delivering a solution comprising at least one solvent and at least one solute to tissues and/or organs. The method comprises the steps of providing a device as described above; disrupting the separation means thereby obtaining the solution wherein the solute is dissolved in said solvent; and immediately delivering the obtained solution to said tissues and/or organs; wherein said solute is sodium 2-mercaptoethanesulfonate in powder form.

In a preferred embodiment, the solution is delivered in droplets form by applying a manual pressure on pressure means 58 and thereby on the fluidly connected chambers wall. In this embodiment, the control means of the device comprise the pressure means 58. The droplets have a predetermined volume which is of from 50 to 200 preferably from 60 to 150 more preferably from 70 to 100 In this way the user is capable of hand manipulating the device for directly delivering the Mesna solution from the device to the desired location and in desired volumes. The design, the manufacture and the use of the device are simple thereby saving costs and working time. Furthermore, the device offers a possibility to control the amount of fluid flowing out of the device, thereby avoiding any excess of delivered solution volume. In addition, the user will be provided with a control over the time at which the fluid is flowing out of the device. These possibilities are not offered by the devices of the prior art.

Referring to FIG. 7A, a fourth embodiment of the device is shown. The device comprises a single chamber 109 for housing the solute, a spring piston 108, an outlet 101 and a connection means 103. The outlet 101 is suitable to be in fluid communication with the single chamber 109 of the device and can be covered by a removable cap or a foil 101′ when the device is not used. Said device can have any shape and is preferably cylindrical as shown in FIG. 7A. The connection means 103 can be an opening or a tube positioned at the outer wall of the device. The connection means 103 can be covered by a removable cap or a foil 102 when the device is not used. Said connection means 103 is in fluid communication with the single chamber of said device.

Preferably, the device further comprises a lumen 107 positioned between the single chamber 109 and the outlet tube 106 of the device. Said lumen 107 is movable between an open position in which said lumen 107, the single chamber 109 and the outlet tube 106 are in fluid communication with each other and a closed position (FIG. 7A) in which said lumen 107, single chamber 109 and the outlet tube 106 are not in fluid communication with each other. Movement of the lumen can be ensured by placing said lumen into a pivotable section of the device. Said section is pivotable by manipulating a lever 100 provided to the outer wall of the device as shown in FIG. 7A. Alternatively, the lumen 107 can be movable by simply placing on the outer wall of the device a movable button which mechanically moves said lumen between the open and the closed position. Movement of the lumen can also be performed using a piston valve, a rotary valve or any other means known to the person skilled in the art.

The device is connectable to a solvent source via the connection means 103. Said solvent source can be a second device 104 as shown in FIG. 7A. The connection means 103 connects the second device to the single chamber 109 of the device. The second device 104 is used for injecting the solvent into the single chamber of the device thereby obtaining the desired solution, i.e. Mesna solution, at the desired concentration. The second device 104 is preferably provided with volume graduation indications. Said second device can be a prefilled syringe as shown in FIG. 7A or any other device known by the person skilled in the art. The device according to this embodiment offers the user the possibility to choose the solvent volume to be added to the single chamber of the device and thereby of the concentration of Mesna solution to be used.

The connection means 103 is in fluid contact with the single chamber when the second device is connected to said device. The connection means 103 is provided with a one way valve 110 thereby preventing the content of the single chamber from flowing outside the device when the second device is disconnected.

In use, the user first makes sure that the lumen is in closed position. The user then connects the second device 104 to the device 102 via the connection means 103 as shown in FIG. 7B. The second device 104 comprises the solvent and at least one outlet through which the solvent will be inserted into the single chamber of the device 102. The outlet of the second device is preferably provided with a spring which reinforces the connection between both devices. The user then injects the solvent into the single chamber 109 of the device as shown in FIG. 7B. The injection is performed via the one way valve 110 of the connection means 103. The content of the single chamber will dissolve in the solvent thereby obtaining the Mesna solution. During the injection, the spring piston 108 of the device will be pushed further away from the outlet 101 of the device due to the pressure applied by the solvent entering the single chamber 109. After injection of the desired solvent volume, the second device 104 is disconnected from the device 102. Mesna solution is prevented from flowing out of the connection means due to the presence of the one way valve. The device can be agitated or shacked to further mix the solvent and the solute. Afterwards, the alignment means 100 and thereby the lumen is moved from the closed position to the open position. FIGS. 7C and 7C show the lumen in an intermediate position, i.e. between the closed and the open position. Due to said movement, the lumen, the single chamber and the outlet tube are brought in fluid communication with each other. The user can then start dispensing the solution contained inside the single chamber by pushing the spring pistol towards the outlet of the device. The lumen 107 and the lever 100 can preferably be replaced by a valve, such as a trumpet valve or any other suitable valve known to the person skilled in the art. The outlet of the device can be used for direct delivery of the Mesna solution to the target or can be connected to any other suitable device such as a dissector 115 (FIG. 7A and FIG. 7B).

Preferably the device is provided with control means for controlling the volume of the solution flowing out of the device in such a way that each opening of the lumen or the control valve dispenses a predetermined amount of solution.

In a preferred embodiment, the invention provides a method for weakening inter-tissues and/or organs adhesion by delivering a solution comprising at least one solvent and at least one solute to tissues and/or organs. The method comprises the steps of providing a device as described above; moving the lumen of the device from an open position to a closed position thereby interrupting the fluid connection between the single chamber, the lumen and the outlet of the device; connecting a second device containing the solvent to the device via the connection means; injecting a solvent volume into the single chamber of the device thereby dissolving the solute in the solvent and obtaining the solution; disconnecting said second device from the device; moving the lumen from the closed position to the open position thereby creating a fluid connection between the single chamber, the lumen and the outlet of the device; and delivering the obtained solution to said tissues and/or organs by pushing the spring piston towards the outlet of the device. Preferably, the delivery of the solution is performed immediately after obtaining said solution. Preferably, said solute is sodium 2-mercaptoethanesulfonate in powder form.

Referring to FIG. 8A and FIG. 8B, a fifth embodiment of the device is shown. The device comprises a first chamber 202 for housing the solute, i.e. Mesna in powder form; a second chamber 201 for housing the solvent and at least one outlet 206 for delivering the solution. Said outlet is suitable to be in fluid communication with at least one of the chambers. On FIG. 8A and FIG. 8B, the outlet 206 is suitable to be in fluid communication with the second chamber 201. Said outlet can be covered by a removable cap (not shown) when the device is not used. The outlet can be used for direct delivery of the Mesna solution to the target or can be connected to any other suitable device 205.

Preferably, the device further comprises a closable air inlet 204. The air inlet is preferably in fluid communication with the chamber comprising the solvent. In a preferred embodiment, the air inlet is closable using the movable closing means 203. Said closing means is movable from an open position in which the air inlet is open such as air can flow in the solvent chamber (FIG. 8C) to a closed position in which the air inlet is closed such as air is prevented from flowing in or outside the solvent chamber (FIG. 8D). The air inlet can be replaced by a one-way valve.

In a preferred embodiment, the first chamber 202 and the second chamber 201 are assembled after being filled with the required solute and the required solvent respectively. The connection can be ensured by a connecting mechanism such as a snap fit system or any other system known to the person skilled in the art. The connecting mechanism keeps the assembled chambers together to ensure maximum efficiency during transfer of product between said first and second chambers. The device is preferably provided with an outer wall surrounding the chambers. Said outer wall can be a film sealed around the chambers thereby ensuring tightness of the device.

Preferably, the first chamber comprises a bottom wall which is a disruptable membrane and the second chamber comprises an upper wall which is a disruptable membrane. Preferably the chambers are assembled such as the disruptable membrane of the first and the second chamber are in contact with each other thereby creating a double barrier 213 also called separation means. This means that the chambers are separated from each other by a double barrier 213. Preferably, the disruptable membranes are made of aluminium laminate.

Preferably, the first chamber 202 is provided with a pressure means 210. Said pressure means is preferably a flexible half cylindrical wall as shown in FIG. 8B. The pressure means is provided with a piercing means 211. The piercing means can be any means known to the person skilled in the art. The pressure means serves the two purposes: breaking the double barrier 213 for reconstituting the solution and expelling the solution out of the device. The pressure means is connected with the piecing means such as when pressing the pressure means, the double barrier 213 is disrupted thereby merging the content of both chambers. The solution is expelled out of the device by applying a manual pressure on said pressure means when the device is in use.

In a preferred embodiment, the solution is delivered in droplets form by applying a manual pressure on pressure means 210. In this embodiment, the control means of the device comprise the pressure means 210. The droplets have a predetermined volume which is of from 50 to 200 preferably from 60 to 150 more preferably from 70 to 100 In this way the user is capable of hand manipulating the device for directly delivering the Mesna solution from the device to the desired location and in desired volumes. The design, the manufacture and the use of the device are simple thereby saving costs and working time. Furthermore, the device offers a possibility to control the amount of fluid flowing out of the device, thereby avoiding any excess of delivered solution volume. In addition, the user will be provided with a control over the time at which the fluid is flowing out of the device. These possibilities are not offered by the devices of the prior art.

Preferably, the remaining first chamber walls and/or the second chamber walls are made of a rigid non-deformable material. Non-deformable material refers to a material that retains its shape when subjected to manual pressure. The non-deformable rigid material allows the first chamber to maintain its intact structure when the chamber bottom wall is being disrupted. The first chamber walls can be made of a plastic material such as a copolymer having hermetic properties allowing a stable and durable conservation of the solute. The rigidity of material also allows an optimal lifetime of the first chamber.

Preferably, said first chamber is provided with a removable cap (not shown) and is preferably provided with a round top. The cap might have a U shape and is produced in a plastic material such as a copolymer. The inner walls of the cap might be threaded in order to be able to screw the cap on the body of the device.

In use, the user makes sure that the air inlet 204 is in closed position. The user removes the cap protecting the first chamber and depresses the pressure means. This leads to the disruption of the double barrier. The content of the first and the second chambers will merge to obtain the solution in which the solute is dissolved in the solvent. The device can be further agitated or shacked to ensure dissolving the solute in the solvent. The air inlet is then opened. The solution can be delivered to the desired location by further applying a pressure on the pressure means acting as a pump of the device as shown in FIG. 8E. Air will be inserted into the second chamber to compensate for the pressure difference inside said chamber after pressing the pressure means and delivering the solution outside the device.

In a preferred embodiment, the invention provides a method for weakening inter-tissues and/or organs adhesion by delivering a solution comprising at least one solvent and at least one solute to tissues and/or organs. The method comprises the steps of providing a device as described above; disrupting the separation means thereby obtaining the solution wherein the solute is dissolved in the solvent; and delivering the obtained solution to said tissues and/or organs; wherein said solute is sodium 2-mercaptoethanesulfonate in powder form. Preferably, the solution is delivered immediately after being obtained in the device.

The present invention further provides for the use of a mucolytic agent in hydrosurgery. Said mucolytic agent is sodium 2-mercaptoethanesulfonate, preferably in powder form. The use of the device and hence the use of a mucolytic agent, i.e. Mesna, in hydrosurgery presents a major advantage. In conventional hydrosurgery methods, it is difficult to apply a pressure which is at the same time sufficient to overcome inter-tissues and/or organs adhesion while avoiding damaging the non-pathologic tissues or organs. The use of Mesna in hydrosurgery allows the use of a lower water pressure while performing the required action, i.e. overcoming inter-tissues and/or inter-cellular adhesion while at the same time preserving the remaining tissues or organs from any damage.

The invention further provides a method and a kit for overcoming inter-tissues and/or inter-cellular adhesion by delivering a solution comprising at least one solvent and at least one solute to tissues and/or organs. The method is preferably a hydrosurgery method comprising the steps of:
a—connecting a first container comprising the solute to a second container comprising the solvent,
b—transferring at least partially the content of the first container into the second container thereby dissolving the solute in the solvent and obtaining the solution. Preferably, the content of the first container is totally transferred to the second container.
c—disconnecting the first container from the second container, and
d—delivering the solution obtained in the second container to said tissues and/or organs,

wherein said solute is sodium 2-mercaptoethanesulfonate in powder form. In a preferred embodiment, the solvent is saline water wherein the content of NaCl is adjusted to make the solution isotonic. Preferably, delivering the solution is performed immediately after obtaining said solution. Preferably, the method further comprises the step of connecting the container comprising the obtained solution to a delivery control device.

It is to be understood that “solvent reservoir” and “solvent container” are used herein as synonyms.

The different steps of the method are shown in FIG. 9A to FIG. 9G. In a preferred embodiment, the first container 252 containing the solute before being connected to the second container 254 is sealed by a disruptable membrane. Said disruptable membrane is preferably disrupted using a disrupting device enclosed in a blister pack 250. The disrupting device is preferably provided with a piercing means which is preferably covered by a removable foil 251.

In a preferred embodiment, the first container 252 is connected to the second container 254 using at least one connection means 256 such as a luer port which is fixed to the first or to the second container.

In a preferred embodiment, the solvent is at least partially transferred in to the first container 252 thereby dissolving the solute in the transferred solvent volume. Said transferred solute volume in which the solute is dissolved is then transferred back into the second container 254 containing the remaining volume of the solvent; this ensures the transfer of the complete amount of the solute from the first container 252 into the second container 254. These steps can be repeated if necessary.

After disconnecting the first container 252, the second container 254 containing the desired solution is connected to a delivery control device for delivering the solution to the target. Said delivery control device is preferably a high pressure pump. The second container can be provided with an attachment means 255 for removably connecting the delivery control device.

In a preferred embodiment, the first container comprises a high amount of solute. Said amount is from 50 to 600 g of Mesna powder, preferably from 80 to 500 g, more preferably from 90 to 400 g, most preferably from 100 to 350 g of Mesna powder or any value comprised in the mentioned ranges. Preferably, the second container comprises a solvent volume of from 500 ml to 8 L, preferably from 1 L to 7 L, more preferably from 1.5 L to 6 L, most preferably from 2 L to 5 L or any volume comprised within the mentioned ranges.

In a preferred embodiment, the method further comprises the steps of connecting the reservoir or the second container comprising the Mesna solution to a water jet surgery system, and delivering the Mesna solution to a target location. Said delivery is thereby made at high pressure. The delivery means is selected from the group comprising: a surgical device, a high pressure pump, a delivery tube, an applicator or any combination thereof. An embodiment of such connection is shown in FIG. 10 showing the reservoir 301 containing the Mesna solution which is connected to a water jet surgery system. Said system comprises a surgical device 302, an electrically driven mechanical system 300, a pedal 303 connected to said electrically driven mechanical system 300. Preferably a waste container 304 is connected to the surgical device 302 for collecting waste produced during operation.

The present invention further provides a kit comprising at least one first container which is sealed by a disruptable membrane and comprising the solute, at least one second container comprising the solvent and at least one disrupting device. The kit further comprises a leaflet provided with instructions to the user and/or information on the solute and/or the solvent. The first container, the second container, the solvent and the solute are as described above. Said solute is sodium 2-mercaptoethanesulfonate in powder form. In a preferred embodiment, the solvent is saline water wherein the content of NaCl is adjusted to make the solution isotonic.

The present invention further provides for the use of a device and/or method according to any embodiment of the invention, for delivering a solution comprising at least one solvent and at least one solute to a surface wherein said solute is a mucolytic agent in powder form. Preferably, said mucolytic agent is Mesna in powder form. Preferably, the solution consists of one solute and one solvent.

WO 2009/022430 A1 describes a skin whitening agent having an excellent whitening effect, an excellent moisture-retaining action (or moisturizing effect), less side effects and high safety is provided. The skin whitening agent contains a mercaptosulfonic acid or a salt thereof as an effective ingredient. The mercaptosulfonic acid may be a mercaptoalkanesulfonic acid or a dimer thereof, and the salt of the mercaptosulfonic acid may be a metal salt (e.g., mesna, and dimesna). The content of the mercaptosulfonic acid or the salt thereof in the skin whitening agent may be about 0.01 to 50% by weight in terms of a solid content. The skin whitening agent is capable of inhibiting melanogenesis. The skin whitening agent may be used in a dosage form for oral administration or external application.

WO 2015/071910 A1 discloses methods of preventing, ameliorating and/or treating radiation induced gastrointestinal tract injury comprising the administration of a therapeutically effective amount of Mesna to a patient are provided. Methods for preventing, treating and/or ameliorating gastrointestinal tract injury induced by radiation therapy, alone or in combination with other therapies for diseases or conditions such as gastrointestinal malignancies, urogenital malignancies, gynecologic malignancies, and osteogenic and other sarcomatous malignancies in which pelvic structures are involved. Auxiliary components such as tablet disintegrants, solubilisers, preservatives, antioxidants, surfactants, viscosity enhancers, coloring agents, flavoring agents, pH modifiers, sweeteners or taste-masking agents may be incorporated into a composition according to WO 2015/071910 A1.

Peter, C. et al. (2000): “Pharmacokinetics and organ distribution of intravenous and oral methylene blue”, European Journal of Clinical Pharmacology, Springer Verlag, DE, vol. 56, no. 3, pages 247-250, describes a determination of the pharmacokinetics and organ distribution of i.v. and oral methylene blue, which is used to prevent ifosfamide-induced encephalopathy in oncology. The concentration of methylene blue in whole blood was measured using high-performance liquid chromatography in seven volunteers after i.v. and oral administration of 100 mg methylene blue with and without mesna. The distribution of methylene blue in different tissues was measured in rats after intraduodenal and i.v. application. The time course of methylene blue in whole blood after i.v. administration showed a multiphasic time course with an estimated terminal half-life of 5.25 h. Following oral administration, the area under the concentration-time curve was much lower (9 nmol/min/ml vs 137 nmol/min/ml). Co-administration of mesna, which could influence distribution by ion-pairing, did not alter the pharmacokinetics. The urinary excretion of methylene blue and its leucoform was only moderately higher after i.v. administration (18% vs 28% dose). Intraduodenal administration to rats resulted in higher concentrations in intestinal wall and liver but lower concentrations in whole blood and brain than i.v. methylene blue. Differences in organ distribution of methylene blue are mainly responsible for the different pharmacokinetics after oral and i.v. administration. If methylene blue acts in the liver, where ifosfamide is primarily activated to reactive and potentially toxic metabolites, oral and i.v. methylene blue are likely to be equally effective. However, if the site of action is the central nervous system, i.v. methylene blue which results in much higher concentrations in brain seems preferable.

WO 98/16213 A1 discloses topical formulations containing sodium 2-mercaptoethanesulfonate (MESNA), the use of MESNA for the preparation of pharmaceutical formulations intended to be used in surgical procedures that involve the dissection of tissues of the use of MESNA in said surgical procedures.

WO 2017/157670 A1 describes a device for delivering a Mesna formulation to tissues and/or organs. The device comprises a first chamber comprising Mesna in solid form, a second chamber comprising a buffer and at least one outlet for delivering the Mesna formulation, said outlet is in fluid communication with at least one of the chambers. The chambers are separated from each other by at least one disruptable separation means and are in fluid communication with each other upon disruption of said separation means thereby forming the Mesna formulation. The pH of the buffer comprised in the second chamber is at least 8.5. The invention further provides a process for the preparation of a Mesna formulation. According to WO 2017/157670 A1, the outlet of the device is connectable to at least one second device selected from the group comprising surgical devices, high pressure pumps, delivery tubes, applicators, minimally invasive surgery systems, robot assisted device and low pressure pump.

U.S. Pat. No. 4,191,176 A discloses an enzymatic intralenticular cataract treatment for removal of nuclear cortical and subcapsular regions of the cataractous lens through enzymatic digestion thereof which comprises introduction of a concentrated solution of a trypsin enzyme into the nuclear and cortical regions of a cataractous lens, and after enzymatic digestion removing the liquefied cataractous material. The procedure allows subsequent removal of the nuclear, cortical and subcapsular portions of a cataractous lens through a very tiny incision in the eye and lens capsule, leaving all other structures within the eye intact. Bovine and porcine trypsins are preferred. According to U.S. Pat. No. 4,191,176 A, for an enzyme containing solution injected into the lens, the distribution pattern of the injected fluid may be observed by incorporating a soluble, inert dye such as fluorescein into the injection fluid in amounts of 0.01-1.0% w/v.

U.S. Pat. No. 5,273,751 A describes compositions for killing undifferentiated epithelial cells during cataract surgery on an eye to prevent posterior capsule clouding after the surgery and to a method for performing cataract surgery on an eye including injecting a cell-killing substance between the anterior capsule and the natural lens prior to removing the natural lens from the eye. The cell-killing substance is preferably an acid or base adjusted aqueous solution having a pH in the range between about 1.0 to below 6.5 or about above 7.5 to 14.0; or a hypotonic solution having a salinity less than 0.9% or a pH adjusted hypotonic solution having a salinity less than 0.9% and a pH either below 6.5 or above 7.5. The compositions of U.S. Pat. No. 5,273,751 A also incorporate a viscoelastic material, a dye or a mixture thereof, in combination with the cell-killing substance. According to U.S. Pat. No. 5,273,751 A, for the purpose of visually determining that cell-killing substance has completely filled the pocket and expanded all portions of the anterior capsule segment, a dye is admixed with the cell-killing substance prior to or after injection of the cell-killing substance between the inner surface of the anterior capsule and the lens.

US 2007/016174 A1 discloses a robotic surgical instrument for the control of flows of one or more fluids into and out of a surgical site. The robotic surgical instrument may include a housing, a flow control system, a hollow tube, and one or more hose fittings. The housing to couple the instrument to a robotic arm. The flow control system mounted in the housing includes one or more controlled valves to control the flow of one or more fluids. The hollow tube has a first end mounted in the housing coupled to the flow control system. A second end of the hollow tube has one or more openings to allow the flow of fluids into and out of the surgical site. The hose fittings have a first end coupled to the flow control system and a second end to couple to hoses.

The invention further provides a composition comprising Mesna, wherein the composition further comprises one or more dyes.

In particular, the composition comprising Mesna and one or more dyes is of use in surgery, and more in particular in surgery where Mesna is applied at a cleavage plane between two or more tissues for facilitating tissue separation. The dyes function to stain and, consequently, visualize parts of the tissues on which the Mesna composition is applied. This entails the advantage that a medical practitioner can see the parts where the composition is applied, allowing intuitive and quick ascertaining of a desired application of Mesna to the tissues, or if the application of Mesna should be adjusted, which adjustments can also be directly seen by the visualization brought about by the one or more dyes. The use of a dye allows to visualize whether the mesna solution remains in place for a sufficient duration for effective chemical action. It also helps visualize the cleavage plane, which is very useful to the surgeon. This is of great importance for allowing efficient dissection of tissues facilitated by Mesna, and certainly in view of the knowledge that flooding a surgical cavity in order to make sure that all tissues to be dissected are wetted by Mesna is not an option, because the amount of Mesna which can be used in a single surgical procedure is limited by toxicity considerations.

In a preferred embodiment, the one or more dyes are selected from the group consisting of indocyanine green, riboflavin, riboflavin 5′-phosphate sodium, methylene blue, Blue Patented V, indocyanine green and fluorescein.

Indocyanine green is a fluorescent cyanine dye with a peak spectral absorption at about 800 nm, i.e. it is a green-colored dye. For use in combination with Mesna, indocyanine green shows the advantage of acting as a reductant, thus not reacting with Mesna which is prone to oxidation. Besides, the green color has the advantage of being especially visible on tissues.

As a fluorescent dye, riboflavin provides a yellow to orange-yellow coloring to substrates stained with it. For use in combination with Mesna, riboflavin shows the advantage of acting as a reductant, thus not reacting with Mesna which is prone to oxidation.

Riboflavin 5′-phosphate sodium is the phosphate sodium salt of riboflavin. Like riboflavin, riboflavin 5′-phosphate sodium provides a yellow to orange-yellow coloring to substrates stained with it. Like riboflavin, for use in combination with Mesna, riboflavin 5′-phosphate shows the advantage of acting as a reductant, thus not reacting with Mesna which is prone to oxidation.

Methylene blue is a fluorescent thiazine dye with a blue color. Methylene blue acts as an oxidant, yet by intelligent selection of relative amounts of methylene blue in respect of Mesna, oxidation of Mesna by methylene blue can be rendered negligible.

Fluorescein is a red-colored fluorophore which appears purple under UV light. When applying UV light, fluorescein is thus well-suited for visualizing a composition comprising Mesna on tissues.

The five dyes described above are approved for administration to humans as a pharmaceutical product, e.g. as a contrast product, nutrient, disinfectant, etc.

In a preferred embodiment, the composition comprises one or more pharmaceutically acceptable solvents and/or one or more pharmaceutically acceptable buffers, the one or more solvents and/or one or more buffers comprising from 0.2 weight per volume percent to 30 weight per volume percent, more preferably from 0.4 weight per volume percent to 25 weight per volume percent, more preferably from 2 weight per volume percent to 22 weight per volume percent, yet even more preferably from 3 weight per volume percent to 14 weight per volume percent, yet even more preferably from 4 weight per volume percent to 6 weight per volume percent of Mesna and from 0.0001 weight per volume percent to 30 weight per volume percent, more preferably from 0.002 weight per volume percent to 10 weight per volume percent, more preferably from 0.005 weight per volume percent to 1 weight per volume percent, even more preferably from 0.008 weight per volume percent to 0.5 weight per volume percent and even more preferably from 0.01 weight per volume percent to 0.1 weight per volume percent of the one or more dyes.

Said weight percentages of the one or more dyes are large enough to allow sufficient staining and clear visualization of tissues on which a composition comprising Mesna and one or more dyes is applied while not too large so that excessive use or wastage of the one or more dyes occurs or that undesired damage to the tissues would take place because of the one or more dyes.

When used in surgery, said weight percentages of Mesna are high enough for facilitating tissue separation due to breakage of molecular bonds between tissue layers by Mesna, in particular by Mesna breaking disulfide bonds of polypeptide chains and proteins which disulfide bonds are responsible for the adherence of pathological tissues and for the strength of fibrosis, and are low enough for minimizing the risk of undesired tissue separation of surrounding tissues which are not to be separated.

In addition, said weight percentages of Mesna and of the one or more dyes allow for complete dissolving in the one or more pharmaceutically acceptable solvents and/or one or more pharmaceutically acceptable buffers, and resulting solutions comprising Mesna and the one or more dyes according to said weight percentages show good stability properties.

In a preferred embodiment, the one or more pharmaceutically acceptable solvents are selected from the group comprising water, preferably sterile water, and a solution of up to 0.9 mass/volume percent of NaCl in water, preferably a sterile solution of up to 0.9 mass/volume percent of NaCl in water.

In a preferred embodiment, a solution of Na₂HPO₄ in water or a solution of Na₂HPO₄ and NaCl in water is selected as pharmaceutically acceptable buffer. More preferably, the buffer is an aqueous solution of 10 mM Na₂HPO₄ or an aqueous solution of 10 mM Na₂HPO₄ and 75 mM NaCl. Preferably, the water is water of high purity (e.g. water for injection as per pharmaceutical standard) and may contain between 0 and 0.9 mass/volume percent of NaCl. Preferably, the water is sterile.

The invention further provides a composition comprising Mesna, wherein the composition further comprises one or more dyes, for use in a method of surgery. Preferably, said method of surgery comprises the step of separation of tissue layers along a cleavage plane between said tissue layers, wherein the separation of tissue layers is facilitated by applying the composition comprising Mesna and one or more dyes to said cleavage plane. Due to the incorporation of one or more dyes, the Mesna composition is especially suited for visualizing parts of tissues on which Mesna is applied, allowing fine-tuning of surgery involving dissection of tissue layers facilitated by application of Mesna.

The invention further provides a kit comprising Mesna, wherein the kit further comprises one or more dyes.

The kit can be used to allow a user, such as a medical practitioner or an assisting person, to manufacture a composition comprising Mesna and one or more dyes shortly prior to the intended use of the composition comprising Mesna. This minimizes the risk of Mesna oxidation and maximizes Mesna activity for applications of facilitating tissue separation.

In a preferred embodiment of the kit, the one or more dyes are selected from the group consisting of indocyanine green, riboflavin, riboflavin 5′-phosphate sodium, methylene blue, Blue Patented V, indocyanine grre and fluorescein. For the properties, advantages and technical effects of these dyes is referred to the above description.

In a preferred embodiment of the kit, Mesna is in powder form and the kit further comprises one or more pharmaceutically acceptable solvents and/or one or more pharmaceutically acceptable buffers.

A kit comprising one or more dyes and Mesna in powder form next to one or more pharmaceutically acceptable solvents and/or one or more pharmaceutically acceptable buffers allows one or more dyes and Mesna in powder form to be dissolved in the one or more pharmaceutically acceptable solvents and/or one or more pharmaceutically acceptable buffers shortly before using the resulting Mesna solution, thus minimizing the risk of Mesna oxidation and maximizing Mesna activity for facilitating tissue separation, and also minimizing the risk of degradation of the one or more dyes when dissolved in the one or more pharmaceutically acceptable solvents and/or one or more pharmaceutically acceptable buffers.

Preferably, the one or more pharmaceutically acceptable solvents are selected from the group comprising water, preferably sterile water, and a solution of up to 0.9 mass/volume percent of NaCl in water, preferably a sterile solution of up to 0.9 mass/volume percent of NaCl in water.

Preferably, a solution of Na₂HPO₄ in water or a solution of Na₂HPO₄ and NaCl in water is selected as pharmaceutically acceptable buffer. More preferably, the buffer is an aqueous solution of 10 mM Na₂HPO₄ or an aqueous solution of 10 mM Na₂HPO₄ and 75 mM NaCl. Preferably, the water is water of high purity (e.g. water for injection as per pharmaceutical standard) and may contain between 0 and 0.9 mass/volume percent of NaCl. Preferably, the water is sterile.

In a preferred embodiment, the kit further comprises a fluorescence surgical goggle. Any goggle suitable for making fluorescence visual to a user, as known in the state of the art, may be selected as the fluorescence surgical goggle. According to an embodiment, the fluorescence surgical goggle comprises an excitation light source, and preferably a multi-wavelength excitation light source, a camera equipped with a fluorescence filter for collecting fluorescence while eliminating interference from the excitation light, a head mount display, and a computer configured for image processing and/or other imaging processing accessories. A graphical programming software program may be used to fuse a background image with fluorescence emission captured by the camera, and to project to the head mount display for real-time image guidance. A fluorescence surgical goggle provides the advantage of being able for excitation of fluorescent dyes by directing a light source with a wavelength, equal to an optimal excitation wavelength of the fluorescent dye, to the fluorescent dye, which goggle is simultaneously able to visualize the fluorescence selectively to a user, preferably a medical practitioner, wearing the goggle. According to a preferred embodiment, the kit comprises Mesna, one or more fluorescent dyes, a fluorescence surgical goggle, and optionally one or more pharmaceutically acceptable solvents and/or optionally one or more pharmaceutically acceptable buffers. Accordingly, a user, preferably a medical practitioner, of the goggle can selectively see the fluorescent dye without interferences of naturally occurring colors of the tissues. Preferably, the one or more fluorescent dyes are selected from the group comprising of indocyanine green, riboflavin, riboflavin 5′-phosphate sodium, methylene blue and fluorescein.

The invention further provides a method for visualization of a composition comprising Mesna which is applied to tissues, comprising the step of applying the composition comprising Mesna to two or more tissues, wherein the composition further comprises one or more dyes for visualizing parts of the two or more tissues where the composition has been applied.

In particular, the method for visualization of a composition comprising Mesna which is applied to tissues is of use in surgery, and more in particular in surgery where Mesna is applied at a cleavage plane between two or more tissues for facilitating tissue separation. The dyes function to stain and, consequently, visualize parts of the tissues on which the Mesna composition is applied. This entails the advantage that a medical practitioner can see the parts where the composition is applied, allowing intuitive and quick ascertaining of a desired application of Mesna to the tissues, or if the application of Mesna should be adjusted, which adjustments can also be directly seen by the visualization brought about by the one or more dyes. The use of a dye allows to visualize whether the mesna solution remains in place for a sufficient duration for effective chemical action. It also helps visualize the cleavage plane, which is very useful to the surgeon. This is of great importance for allowing efficient dissection of tissues facilitated by Mesna, and certainly in view of the knowledge that flooding a surgical cavity in order to make sure that all tissues to be dissected are wetted by Mesna is not an option, because the amount of Mesna which can be used in a single surgical procedure is limited by toxicity considerations.

In a preferred embodiment of the method, the one or more dyes are selected from the group consisting of indocyanine green, riboflavin, riboflavin 5′-phosphate sodium, methylene blue, Blue Patented V, indocyanine green and fluorescein. For the properties, advantages and technical effects of these dyes is referred to the above description.

In a preferred embodiment of the method, the one or more dyes are one or more fluorescent dyes and wherein the method further comprises the step of providing a fluorescence surgical goggle to enable visualization of the fluorescent dyes through the goggle. Accordingly, a user, preferably a medical practitioner, of the goggle can selectively see the fluorescent dye without interferences of naturally occurring colors of the tissues. Preferably, the one or more fluorescent dyes are selected from the group comprising of indocyanine green, riboflavin, riboflavin 5′-phosphate sodium, methylene blue and fluorescein.

The invention further provides a use of a composition comprising Mesna, wherein the composition further comprises one or more dyes, in said method for visualization. Accordingly, all the technical achievements and advantageous characteristics of the composition comprising Mesna, wherein the composition further comprises one or more dyes, are combined with those of the method for visualization.

The invention further provides a composition comprising Mesna for use in a method of robot-assisted surgery.

A composition comprising Mesna for use in a method of surgery shows the advantages that the surgery is facilitated by breakage of molecular bonds between tissue layers by application of Mesna to a targeted cleavage plane between the tissue layers. Moreover, when applying Mesna, cleavage planes between tissues are revealed. Accordingly, a very precise and selective mechanical detachment of pathological tissues is possible without cutting. This advantageous manner of mechanical detachment of tissues following breakage of molecular bonds by Mesna can be construed as chemically assisted tissue dissection. A method of surgery without the use of Mesna would require more effort in separating tissue layers from another, resulting in a longer duration of surgery or even undesired tissue damage. In particular, without the use of Mesna, dissection would have to be performed by electric or mechanical knives with increased risk of cutting accidentally or unavoidably critical organs. Chemically assisted tissue dissection also facilitates “en bloc” dissection of non-infiltrating tumors, with less risks of damaging underlying tissues or leaving behind tumor material.

The use of a composition comprising Mesna in a method of robotic assisted surgery shows the advantages that simultaneously Mesna can be applied very precisely to a targeted cleavage plane between tissue layers to be separated from another. Moreover, when applying Mesna, cleavage planes between tissues are revealed. The application of a composition containing Mesna can also facilitate the dissection of fibrosis.

Accordingly, a more precise mechanical detachment of pathological tissues is possible without cutting. This advantageous cooperation between Mesna as a chemical compound facilitating tissue dissection and robotic assisted surgery can be construed as both robotic and chemically assisted tissue dissection. Manual application of Mesna, without a computer-assisted electromechanical device, on the one hand, would not allow such precise application of Mesna, resulting in a less precise separation of tissue layers. In particular, manual application of Mesna would require a separate surgical tool, such as a cannula, operated by a medical practitioner separately or through an additional trocar without direct coordination with a robot-assisted electromechanical device and could be insufficiently precise or could result in excessive spillage of Mesna. A method of robotic assisted surgery without the use of Mesna, on the other hand, would require more effort in separating tissue layers from another, resulting in a longer duration of surgery or even undesired tissue damage. In particular, without the use of Mesna, dissection would have to be performed by electric or mechanical knives with increased risk of cutting accidentally or unavoidably critical organs.

Preferably, the composition comprising Mesna is sterile. This is of particular importance in the current context of the preparation of a composition comprising Mesna for use in a method of surgery, in order to avoid infections during surgery.

In a preferred embodiment, the method of surgery is selected from the group comprising skull base surgery, open surgery, non-invasive surgery, thoracic surgery, cardiovascular surgery, dentistry, orthopedic surgery, plastic surgery, ophthalmic surgery, neck surgery, neurosurgery, maxillo-facial surgery, gallbladder surgery, endoscopic surgery, laparoscopic surgery, vascular surgery, colorectal surgery, general surgery, gynecologic surgery, heart surgery, endometriosis, head surgery, neck surgery, transoral neck surgery, transoral head surgery and urologic surgery.

In a preferred embodiment, the composition comprises 1% by weight to 30% by weight, more preferably 2% by weight to 22% by weight, yet even more preferably 3% by weight to 14% by weight, yet even more preferably 4% by weight to 6% by weight of Mesna, supplemented up to 100% by weight with one or more pharmaceutically acceptable solvents and/or one or more pharmaceutically acceptable buffers, based on the overall weight of the composition. Compositions according to said weight percentages allow for complete solubilization of Mesna in the one or more pharmaceutically acceptable solvents and/or one or more pharmaceutically acceptable buffers. Also, said concentrations of Mesna in the composition are high enough for facilitating tissue separation and are low enough to avoid excessive wastage of Mesna in a body of a patient where not necessary, thereby not exceeding a maximum amount of Mesna acceptable to prevent systemic potential toxicity.

In a preferred embodiment, the one or more pharmaceutically acceptable solvents are selected from the group comprising water, preferably sterile water, and a solution of 0.9 mass/volume percent of NaCl in water, preferably a sterile solution of 0.9 mass/volume percent of NaCl in water.

In a preferred embodiment, a solution of Na₂HPO₄ in water or a solution of Na₂HPO₄ and NaCl in water is selected as pharmaceutically acceptable buffer. More preferably, the buffer is an aqueous solution of 10 mM Na₂HPO₄ or an aqueous solution of 10 mM Na₂HPO₄ and 75 mM NaCl. Preferably, the water is water of high purity (e.g. water for injection as per pharmaceutical standard) and may contain between 0 and 0.9 mass/volume percent of NaCl. Preferably, the water is sterile.

In a preferred embodiment, the composition comprises one or more chelating agents.

In a preferred embodiment, edetate disodium is selected as chelating agent.

According to embodiments, preservation agents such as benzyl alcohol may be added to the composition comprising Mesna. However, when storing Mesna in powder form and only dissolving Mesna prior to its use in a method of surgery, the use of preservation agents can be avoided.

In a preferred embodiment the composition comprising Mesna further comprises one or more dyes in order to make the topical deposition visible by the surgeon operating the mechanical dissectors. In this way the extent of coverage of the tissues and the cleavage planes can be made more easily identified, which greatly facilitates the dissection.

In a further preferred embodiment, said one or more dyes are selected from the group consisting of indocyanine green, riboflavin, riboflavin 5′-phosphate sodium, methylene blue and fluorescein.

In a preferred embodiment, the dye is selected among substance visible in fluorescence or epifluorescence.

The invention further provides a method for producing a composition comprising Mesna for use in a method of robotic assisted surgery, wherein at most one day, more preferably at most 12 hours, even more preferably at most 8 hours, even more preferably at most 6 hours, yet even more preferably at most 4 hours, yet even more preferably at most 3 hours, yet even more preferably at most 2 hours prior to performing the method of surgery, the composition is prepared by the step of dissolving Mesna in powder form in one or more pharmaceutically acceptable solvents and/or one or more pharmaceutically acceptable buffers.

The measure of preparing the composition by dissolving Mesna in powder form in one or more pharmaceutically acceptable solvents and/or one or more pharmaceutically acceptable buffers, at most one day prior to performing the method of surgery, has the advantage that the prepared composition comprising Mesna can be used shortly after dissolving Mesna in powder form, thereby providing maximal Mesna activity for facilitating tissue separation when used in surgery. This maximal Mesna activity can be explained by the high sensitivity of a liquid solution of Mesna, for example of a liquid solution of Mesna in water, to oxidation. Dissolving Mesna in powder form shortly before use thus minimizes Mesna oxidation and as a result maximizes Mesna activity and alleviates the need for chelating and antibacterial agents. Maximization of Mesna activity is advantageous for use of Mesna in a method of surgery. The measure of preparing the composition by dissolving Mesna in powder form in one or more pharmaceutically acceptable solvents and/or one or more pharmaceutically acceptable buffers, at most one day prior to performing the method of surgery, also has the advantage that a composition comprising a tailor-made concentration of Mesna and volumes of the composition comprising Mesna can be prepared case-by-case in function of the needs for a method of surgery by dissolving a particular amount of Mesna in powder form in a particular amount of one or more pharmaceutically acceptable solvents and/or one or more pharmaceutically acceptable buffers. The use of tailor-made Mesna concentrations and volumes of compositions comprising Mesna is advantageous for facilitating tissue separation during surgery.

Additionally, the measure of preparing the composition by dissolving Mesna in powder form in one or more pharmaceutically acceptable solvents and/or one or more pharmaceutically acceptable buffers, at most one day prior to performing the method of surgery, appears to be specially advantageous when comparing with common uses of Mesna as known from the state of the art. Most pure Mesna dosage forms used until today are liquid formulations. Since Mesna is very sensitive to oxidation and reacts in the presence of oxygen to form di-Mesna, the aqueous solutions have to be protected against oxygen. In addition, Mesna in liquid form is highly prone to oxidation in presence of metals. Therefore, Mesna solutions are usually sealed into glass ampoules, preferably in low iron glass containers under nitrogen blanket with stabilizers, anti-oxidants and metals chelating agents. Aseptic filling of said glass containers under nitrogen blanket is rather expensive. Furthermore, in some procedures, Mesna solutions are needed at different concentrations from those readily available in commercial glass vials, making the use of said vials tedious or inadequate. When being used, the practitioner has to transfer the solution from glass containers to delivery device or to tube in order to incorporate the solution in the desired application. This step increases the chances of oxidation and contamination of the Mesna solution thereby having reduced Mesna activity or reduced sterility when used, for example in chemically assisted surgery.

In a preferred embodiment, the step of dissolving is performed at most 1 hour, more preferably at most 45 minutes, even more preferably at most 30 minutes, even more preferably at most 20 minutes, even more preferably at most 15 minutes, even more preferably at most 10 minutes, yet even more preferably at most 5 minutes prior to performing the method of surgery. Accordingly, Mesna activity for facilitating tissue separation when used in surgery is maximized.

In a preferred embodiment of a method for producing a composition comprising Mesna for use in a method of surgery, the Mesna in powder form is sterile. In a preferred embodiment, the Mesna in powder form is lyophilized Mesna. In a preferred embodiment, the sterilized Mesna powder is obtained by drying, a sterilized Mesna solution. According to preferred embodiments, said Mesna solution is sterilized by microfiltration or ultrafiltration. Sterility of the Mesna in powder form is desired, and especially in the current context of the preparation of a composition comprising Mesna for use in a method of surgery, in order to avoid infections during surgery.

In a preferred embodiment of a method for producing a composition comprising Mesna for use in a method of surgery, the Mesna in powder form is lyophilized Mesna. Lyophilized Mesna has improved stability and shelf-life compared to technical Mesna. Technical Mesna is Mesna obtained as a stable crystalline powder from synthesis, but this form is not sterile and therefore not suitable to medical applications. In a preferred embodiment, the Mesna in powder form is sterile lyophilized Mesna. In preferred embodiment, the sterilized lyophilized Mesna is obtained by freeze-drying or lyophilizing a sterilized Mesna solution. According to preferred embodiments, said Mesna solution is sterilized by microfiltration or ultrafiltration.

In another preferred embodiment, the Mesna crystalline powder obtained by synthesis is placed in a container and sterilized by terminal gamma ionizing radiation, preferably a ionizing radiation of a dose between 25 and 45 Gy. In a further preferred embodiment, said container is a cartridge of the invention. It should be understood by a skilled person that other conditions of gamma ionizing radiation could be used to sterilize the Mesna powder in a container, without departing from the scope of the present invention.

In a preferred embodiment of a method for producing a composition comprising Mesna for use in a method of surgery, the one or more pharmaceutically acceptable solvents are selected from the group comprising water, preferably sterile water, and a solution of 0.9 mass/volume percent of NaCl in water, preferably a sterile solution of 0.9 mass/volume percent of NaCl in water.

In a preferred embodiment of a method for producing a composition comprising Mesna for use in a method of surgery, a solution of Na₂HPO₄ in water or a solution of Na₂HPO₄ and NaCl in water is selected as pharmaceutically acceptable buffer. More preferably, the buffer is an aqueous solution of 10 mM Na₂HPO₄ or an aqueous solution of 10 mM Na₂HPO₄ and 75 mM NaCl. Preferably, the water is water of high purity (e.g. water for injection as per pharmaceutical standard) and may contain between 0 and 0.9 mass/volume percent of NaCl. Preferably, the water is sterile.

In a preferred embodiment of a method for producing a composition comprising Mesna for use in a method of surgery, one or more chelating agents are added to the one or more pharmaceutically acceptable solvents and/or one or more pharmaceutically acceptable buffers.

In a preferred embodiment of a method for producing a composition comprising Mesna for use in a method of surgery, edetate disodium is used as chelating agent. However, when storing Mesna in powder form and only dissolving Mesna prior to its use in a method of surgery, the use of chelating agents for binding metals can be avoided, since Mesna oxidation is avoided.

According to embodiments, preservation agents such as benzyl alcohol may be added to the one or more pharmaceutically acceptable solvents and/or one or more pharmaceutically acceptable buffers. However, when storing Mesna in powder form and only dissolving Mesna prior to its use in a method of surgery, the use of preservation agents can be avoided, since Mesna oxidation is avoided.

The invention further provides a kit for obtaining a composition comprising Mesna for use in a method of robot assisted surgery, wherein the kit comprises Mesna in powder form next to one or more pharmaceutically acceptable solvents and/or one or more pharmaceutically acceptable buffers.

A kit comprising Mesna in powder form next to one or more pharmaceutically acceptable solvents and/or one or more pharmaceutically acceptable buffers allows Mesna in powder form to be dissolved in the one or more pharmaceutically acceptable solvents and/or one or more pharmaceutically acceptable buffers shortly before using the resulting Mesna solution, thus minimizing the risk of Mesna oxidation and maximizing Mesna activity for facilitating tissue separation when used in surgery.

In a preferred embodiment of the kit, the one or more pharmaceutically acceptable solvents are selected from the group comprising water, preferably sterile water, and a solution of 0.9 mass/volume percent of NaCl in water, preferably a solution of 0.9 mass/volume percent of NaCl in water.

In a preferred embodiment of the kit, a solution of Na₂HPO₄ in water or a solution of Na₂HPO₄ and NaCl in water is selected as pharmaceutically acceptable buffer. More preferably, the buffer is an aqueous solution of 10 mM Na₂HPO₄ or an aqueous solution of 10 mM Na₂HPO₄ and 75 mM NaCl. Preferably, the water is water of high purity (e.g. water for injection as per pharmaceutical standard) and may contain between 0 and 0.9 mass/volume percent of NaCl. Preferably, the water is sterile.

In a preferred embodiment, the kit comprises one or more chelating agents.

In a preferred embodiment of the kit, edetate disodium is selected as chelating agent.

The invention further provides a robot instrument for chemically assisted mechanical dissection comprising:
a cartridge comprising a composition comprising Mesna in a liquid form;
a pump connectable, and preferably connected, to said cartridge in order to feed the composition comprising Mesna to an outlet tubing of said pump, upon actuation of said pump;
a robot arm comprising an irrigation channel with an inlet connectable, and preferably connected, to said outlet tubing of said pump and comprising an outlet connectable to an irrigation tip mountable, and preferably mounted on said robot arm, wherein said tip comprises at least one surgical tool selected from the group comprising scalpels, scissors, bovies, forceps, dissector, elevators, hooks, probes, needles, knot pushers, retractors, scopes, clamps and graspers and wherein the tip further comprises at least one irrigation orifice on said at least one surgical tool, said irrigation orifice fluidly connected to the irrigation channel;

whereby actuation of the pump supplies composition comprising Mesna from the cartridge through the irrigation channel to said irrigation tip of the mounted robot arm, and whereby the actuation of the pump is controllable by an operating switch.

Due to the cartridge comprising a composition comprising Mesna in liquid form, the robot instrument for chemically assisted tissue dissection makes use of Mesna for facilitating separation of tissue layers, resulting from breakage of molecular bonds between tissue layers by application of Mesna to a cleavage plane between said tissue layers. Moreover, when applying composition comprising Mesna, cleavage planes between tissues are revealed. Accordingly, a mechanical detachment of pathological tissues is possible without cutting. Without the use of Mesna, more effort would be required in separating tissue layers from another, resulting in a longer duration of surgery or even undesired tissue damage. In particular, without the use of Mesna, dissection would have to be performed by electric or mechanical knives with increased risk of cutting accidentally or unavoidably critical organs. Chemically assisted tissue dissection also facilitates “en bloc” dissection of non-infiltrating tumors, with less risks of damaging underlying tissues or leaving behind tumor material. Chemically assisted tissue dissection also facilitates the dissection of fibrotic tissues

In surgery, the accuracy of topical deposition of Mesna is important for two reasons. Firstly, Mesna must wet a cleavage plane between tissues that are to be dissected. This is not always obvious when a patient is in the Trendelenburg position and the liquid does not flow upwards. For this reason, the outlet of an irrigation channel for providing a composition comprising Mesna must be at the tip of a chemically assisted mechanical dissector placed accurately on the cleavage plane, to ensure proper wetting of the cleave plane with Mesna. Secondly, the amount of Mesna must be limited in order to avoid toxicity effects on the tissues and on the cells. Flooding the surgical cavity in order to make sure that all tissues to be dissected are wetted by Mesna is not an option.

The preferred embodiment of the robot system is shown in FIG. 11.

The cartridge of the robot instrument according to the invention contains Mesna in solid form and a solvent. The Mesna is dissolved in the solvent less than 24 hours, preferably less than 12 hours before use. In a preferred embodiment, a laminate of the said cartridge holds a piercing cylinder which slides into a solvent compartment and leads to a rupturing of a membrane and mixing of a solvent with a Mesna in the solid form to obtain a composition comprising Mesna, whereby said composition is in a liquid form.

In a preferred embodiment, the composition comprising Mesna further comprises at least one dye. In one embodiment, said at least one dye is in a solvent compartment. In another embodiment, dye is in a solid form in a compartment separated from the solvent.

In a preferred embodiment, said at least one dye can be activated in fluorescence. In a further preferred embodiment, said robot instrument is equipped by a fluorescence surgical google. This embodiment allows image assisted surgical operations, which are of high precision and pose less risk to injuring the surrounding tissues and organs. The image assisted surgery allows easier differentiation of the tissues to be treated from the surrounding tissues.

In a preferred embodiment, the fluid connection between the cartridge and the robot arm is made by a disposable tubing, with a connector at each end. In a further preferred embodiment, said connector is a male Luer lock connector.

In a particularly preferred embodiment, the female Luer connection of the cartridge includes a normally closed valve which open upon connection of the male Luer from the tubing.

In a further preferred embodiment, a particle filter is inserted at the inlet side of the tubing. A non-limiting example of such filter is a 5 micron particle filter. It should be understood by a skilled person that any filter suitable for filtering composition comprising Mesna in liquid form may be used in the robot instrument of the invention without departing form the scope of the invention. The filter is intended to retain potential undissolved particles and thus enable the safe application of the Mesna solution.

A peristaltic pump cassette is inserted in the tubing. The cassette is attached to the pump motor before use. The motor is turned on when the foot pedal is depressed by the operator of the robot. In a preferred embodiment, a peristaltic pump cassette is disposable. In a preferred embodiment, a peristaltic pump cassette is sterilized. In a particularly preferred embodiment, the peristaltic pump cassette is sterilized by ionization, application of sterilizing gas and the like.

The speed and the function of a pump motor is controllable by an operator sitting at a console by an operating switch. In a preferred embodiment, said operating switch is a foot pedal placed under the foot of an operator. In a preferred embodiment, the speed of the pump motor is variable. In a particularly preferred embodiment, the whereby the speed of irrigation is controllable by a pump. In a further preferred embodiment, the speed of irrigation can be adjusted between 1 ml/min and 50 ml/min. In a preferred embodiment, the maximum outlet pressure is of the pump 2 bars.

The outlet of the tubing of said peristaltic pump is connected to the gearbox of the robot arm. In a preferred embodiment, the pump motor can be attached to a pole or laid down on a flat surface near the robot.

In a preferred embodiment, the cartridge is hung on a hanger attached to the pump motor.

According to embodiments of the invention, the robot arm robot arm comprises an irrigation channel with an inlet connectable, and preferably connected, to said outlet tubing of said pump and comprises an outlet connectable to an irrigation tip mountable, and preferably mounted on said robot arm. In a preferred embodiment, said tip is mounted on said robot arm.

In a preferred embodiment, said tip mountable, and preferably mounted on said robot arm comprises at least one surgical tool selected from the group comprising scalpels, scissors, bovies, forceps, dissector, elevators, hooks, probes, needles, knot pushers, retractors, scopes, clamps and graspers and wherein the tip of the robot arm further comprises at least one irrigation orifice within said at least one surgical tool.

The arm is comprised of a shaft of suitable length onto which a tip is mountable and preferably mounted, and whereby said shaft is housing the cables for moving the irrigation tip and the fluid channel for dispensing composition comprising Mesna by the irrigation tip and a gear box housing the pulleys connected to the driver motors. In a preferred embodiment, said gear box is connected to electric motors. The gear box is also fitted with a Luer connection for the supply of the composition comprising Mesna.

The fluid channel is preferably provided by extruding a lumen in the stainless-steel shaft in order to leave enough room for the cables between the gear box and the instrument tips. In preferred embodiment, the shaft is electrically isolated. The electrically isolated shaft is suitable for operation in high-frequency mode. In a preferred embodiment, the shaft is isolated electrically by an insulating sheath.

The length of the shaft and the gearbox can be adapted to different types of robots for open or minimally invasive surgeries. Some non-limitive examples of a shaft length are at least 10 cm and at most 45 cm. It should be understood by a skilled person that any suitable shaft length can be used without departing from the scope of the invention.

The irrigation tips are capable of dispensing topically the composition comprising Mesna on the tissues to be dissected while exerting a mechanical separation force, both controlled by the robot operator.

In a preferred embodiment, the irrigation tip mountable and preferably mounted on the robot arm comprises a surgical tool, such as, but not limited to a forceps, dissector, elevator or hook. The function of the elevator of lifting tissues is especially convenient for reaching a cleavage plane between tissues to be treated with a composition comprising Mesna, enabling a proper wetting of the cleavage plane with Mesna. The function of the forceps of grasping tissues is especially convenient for stabilizing and positioning of tissues to be dissected. The dissector is able to interchangeably switch between said latter function and a function of spreading tissues. A hook functions to grab onto tissues and, due to its shape, is especially convenient for stabilizing and positioning of tubular body parts such as ducts and arteries.

In a preferred embodiment, a hook comprising a curved end which is connectable, and preferably connected, to said outlet of said irrigation channel is selected as said surgical tool comprised within a tip mounted on the robot arm. This location of the outlet of the irrigation channel at the curved end of the hook is ideally suited for providing a composition comprising Mesna to a cleavage plane between tissues while the tissues are grabbed by the curved end of the hook.

In a preferred embodiment, depicted in FIG. 13, the irrigation tip mounted on the robot arm is a Maryland type of dissector tip. The Maryland type of dissector used in the robot instrument, preferably comprises two jaws. Said jaws can be moved independently by the robot operator and can be used either in pulling mode or dissection mode.

At least one of the jaws comprises an orifice for dispensing the composition comprising Mesna on the tissues to be dissected before exerting the mechanical separation force. In a preferred embodiment, a diameter of a robot tip orifice is in range from 100 μm-500 μm. In another embodiment, the diameter of a robot tip orifice is adjustable. The diameter of an orifice can influence the mode of irrigation of Mesna. In a preferred embodiment of the invention, the orifice allows for dispensing Mesna in at least two modes. In one preferred embodiment, said mode of Mesna dispensing is a dripping mode. In another preferred embodiment, said mode of Mesna dispensing is a jet mode. The said orifice features allow for a more safe and more precise Mesna application to the surgical cavity. The risks normally connected to Mesna application are minimised, as the flow can be adjusted to the specific nature of the tissues to be treated. Furthermore, the Mesna flow is controllable by an actuation of the pump, and also on a level of an orifice, which can be adjusted to the specific operating procedure to be completed.

The distal part of the robot arm irrigated tip holds at least one surgical tool. In the preferred embodiment, the said distal part holding the jaws of Maryland dissector, whereby said jaws can be rotated independently around the axis.

The proximal part is mounted mechanically to the shaft of the robot arm. It holds the distal piece of the robot arm tip which can be rotated around the axis. The jaws of the Maryland dissector are connected electrically to the metallic shaft by the metallic distal and proximal pieces. At least one of the jaws is connected fluidly to the fluid channel provided in the shaft through the distal and proximal pieces.

In a preferred embodiment, a fluid connection channel of the orifice on the tip mounted onto the robot arm and lumen of the robot arm is provided in the proximal piece of said tip in front of the outlet of the channel for tight fluid connection. Preferably, a groove is provided in the pulley provided in the said proximal piece which is rotating the distal piece around its axis. The fluid channel in the proximal piece has its orifice in front of the groove.

In a preferred embodiment, a fluid connection channel is also provided in the distal piece of the tip. The proximal orifice of this channel is located in front of the groove. In this way, the fluid can pass from the proximal piece to the distal piece. In a preferred embodiment, the fluid tightness is obtained by the flat flanges and the like elements present on the pulleys and the pieces which are closing the groove.

In a preferred embodiment, a groove is provided in the basis of the tip mounted on the robot arm. The distal orifice of the fluid channel provided in the distal piece is placed in front of the groove. The proximal orifice of the fluid channel provided in the irrigated jaw is preferably located in front of the groove. Fluid tightness is obtained by the flat flanges on the jaw basis and the distal piece. The more detailed description of a preferred embodiment of the invention is disclosed in FIGS. 14 and 15.

In a preferred embodiment, the dispensing of the composition comprising Mesna at the orifice of one of the jaws is controlled. The control of the dispensing is achieved by the robot operator by depressing an operating switch, which is preferably a foot pedal placed under the foot of an operator.

It should be understood by a skilled person that the fluid connection with other types of irrigation tips, such as hook dissectors or elevators, is achieved in the above-mentioned manner.

In a further preferred embodiment, the robot arm tip is made of one or more conductive materials connected to a radio frequency source and can also be operated in electric mode for cutting or cauterizing without changing instruments.

In a preferred embodiment, the Mesna irrigated robot arms described above can be used as electrical knives by connecting the shaft to an radio frequent generator in conventional way.

In the preferred embodiment of the invention, by a robot instrument allows additional, or alternative, dissection of tissue layers by application of electrical energy, and also cauterization of tissues by applying electrical energy. Preferably, the dissection or cauterization of tissues by electrical energy is performed by applying heat produced on the surgical tool by the electrical energy. The one or more conductive materials may be selected from any of conductive materials known from the state of the art. The electrical energy providing means may comprise an electrical energy source with a connecting wire for connecting to the surgical tool.

The inclusion of a computer-assisted electromechanical device, which can also be construed as a robotic device, increases the accuracy and precision of said tissue separation facilitated by Mesna, by a more precise and stable positioning of the chemically assisted mechanical irrigation tip by the computer-assisted electromechanical device when compared with manual handling of the dissector. In particular, the at least one steerable robotic arm shows the advantages of being steerable at great precision. The advantageous cooperation between Mesna as a chemical compound facilitating tissue dissection and robotic assisted surgery can be construed as both robotic and chemically assisted tissue dissection. Manual application of Mesna, without a computer-assisted electromechanical device, on the one hand, would not allow such precise application of Mesna, resulting in a less precise separation of tissue layers. In particular, manual application of Mesna would require a separate surgical tool, such as a cannula, operated by a medical practitioner separately or through an additional trocar without direct coordination with a robotic device and could be insufficiently precise or could result in excessive spillage of Mesna. A device for chemically assisted tissue dissection including a robotic device without the use of Mesna, on the other hand, would require more effort in separating tissue layers from another, resulting in a longer duration of surgery or even undesired tissue damage. In particular, without the use of Mesna, dissection would have to be performed by electric or mechanical knives with increased risk of cutting accidentally or unavoidably critical organs.

In a preferred embodiment of the invention, said robot arm and feeding of the composition comprising Mesna is steerable by a computer-assisted electromechanical device by the robot operator. The operator sits behind a control console equipped by a device for a remote computer assisted steering of the robot arms. The control console preferably comprises a control pedals and one or more control sticks for steering of a robot arm. The console is preferably equipped with a stereoscopic oculars, which enable an operator to see a single three-dimensional image. The stereoscopic oculars enable actually viewing the operation cavity in reality, making an accurate simulation of the natural viewing experience and without causing an eye strain and fatigue. The stereoscopic oculars make the operation easier and minimize the risks and injury of the surrounding tissue.

According to a preferred embodiment, the computer-assisted electromechanical device further comprises a console configured for providing instructions to the computer-assisted electromechanical device, preferably for providing instructions to the at least one steerable robotic arm, by means of input provided to the console.

Preferably the robot instrument of the invention comprises manual steering means for manually steering the at least one controllable robotic arm, and wherein the computer-assisted electromechanical device comprises mechanical feedback means configured to connect, and preferably connecting, the at least one controllable robotic arm with the manual steering means.

In a preferred embodiment said robot instrument is equipped by a haptic feedback to an operator. The haptic feedback gives the better overview and control to an operator surgeon, and thus minimizes the risk of injury or dissecting of the surrounding tissue. The haptic feedback means functions for enabling control of force exerted on tissue layers by the surgical tool of the chemically-assisted electromechanical device steered by the at least one robotic arm. Manually steering of the at least one controllable robotic arm can be performed by manual handling of the control means by a medical practitioner.

The use of computer-assisted electromechanical devices, also to be construed as robotic devices or robots, is spreading in many surgical procedures. Most robots are equipped with arms that are equipped with electrical knives and/or mechanical forceps or dissectors, none of which are irrigated. Electrical knives are cutting precisely but not selectively. They would cut indifferently pathologic tissues and organs such as nerves or veins, which do not repair, causing potentially serious side effects. The robot instrument of the invention has the advantages of the computer-assisted electromechanical devices, and at the same time enables using the compositions comprising Mesna as fluids which chemically facilitate the tissue dissection. Thus, the robot instrument of the invention is particularly suitable for complex interventions of a high risk, as it allows better control of dissection and less risk for affecting the surrounding tissue.

The invention further provides a method of robotic-assisted surgery using Mesna, including a step of separation of tissue layers along a cleavage plane between said tissue layers, wherein the separation of tissue layers is facilitated by applying a composition comprising Mesna to said cleavage plane.

Use of Mesna in a method of surgery shows the advantages that the surgery is facilitated by breakage of molecular bonds between tissue layers by Mesna. Moreover, when applying Mesna, cleavage planes between tissues are revealed. Use of Mesna in a method of robotic assisted surgery has the additional advantage that Mesna can be applied very precisely to a targeted cleavage plane between tissue layers to be separated from another. Accordingly, a very precise mechanical detachment of pathological tissues is possible without cutting. This advantageous cooperation between Mesna as a chemical compound facilitating tissue dissection and robotic assisted surgery can be construed as both robotic and chemically assisted tissue dissection. Manual application of Mesna, without a computer-assisted electromechanical device, on the one hand, would not allow such precise application of Mesna, resulting in a less precise separation of tissue layers. In particular, manual application of Mesna would require a separate surgical tool, such as a cannula, operated by a medical practitioner separately or through an additional trocar without direct coordination with a computer-assisted electromechanical device and would not be sufficiently precise or result in excessive spillage of Mesna. A method of surgery or robotic assisted surgery without the use of Mesna, on the other hand, would require more effort in separating tissue layers from another, resulting in a longer duration of surgery or even undesired tissue damage. In particular, without the use of Mesna, dissection would have to be performed by electric or mechanical knives with increased risk of cutting accidentally or unavoidably critical organs. Chemically assisted tissue dissection also facilitates “en bloc” dissection of non-infiltrating tumors, with less risks of damaging underlying tissues or leaving behind tumor material.

According to embodiments of a method of robotic-assisted surgery using Mesna, including a step of separation of tissue layers along a cleavage plane between said tissue layers, wherein the separation of tissue layers is facilitated by applying a composition comprising Mesna to said cleavage plane, the used Mesna may be in the form of a composition comprising Mesna as described in earlier embodiments. Also, such composition comprising Mesna may be prepared according to a method according to earlier embodiments.

The invention is further described by the following non-limiting examples which further illustrate the invention, and are not intended to, nor should they be interpreted to, limit the scope of the invention.

### EXAMPLES

### Examples 1-90: Compositions Comprising Mesna and Selected Dyes

Examples 1-90 relate to compositions comprising Mesna and selected dyes. Each composition according to Examples 1-90 comprises Mesna and a selected dye. In Examples 1-90, weight percentages of Mesna between the different compositions vary from 2% by weight to 22%. The compositions according to Examples 1-15, Examples 16-30, Examples 31-45 and Examples 46-60 are shown in Tables 1-4, respectively. The compositions according to Examples 61-75 and Examples 76-90 are equal to the ones of Examples 1-15 and Examples 31-45, respectively, with the difference that water is replaced by a solution of 0.9 mass/volume percent of NaCl in water. When used in surgery, concentrations of Mesna from 2 weight per volume percent to 22 weight per volume percent, as shown in Examples 1-90, are high enough for facilitating tissue separation due to breakage of molecular bonds between tissue layers by Mesna, in particular by Mesna breaking disulfide bonds of polypeptide chains and proteins which disulfide bonds are responsible for the adherence of pathological tissues and for the strength of fibrosis, and are low enough for minimizing the risk of undesired tissue separation of surrounding tissues which are not to be separated. In fact, flooding a surgical cavity in order to make sure that all tissues to be dissected are wetted by Mesna is not an option.

Water is selected as a pharmaceutically acceptable solvent for Examples 1-15 and 31-45, a 10 mM Na₂HPO₄ aqueous solution is selected as a pharmaceutically acceptable buffer for Examples 16 to 31 and Examples 46 to 60 and a solution of 0.9 mass/volume percent of NaCl in water is selected as a pharmaceutically acceptable solvent for Examples 61-90. A 10 mM Na₂HPO₄ solution is a desired pharmaceutically acceptable buffer in the present context, owing to its solubilizing properties for solubilizing Mesna and the selected dyes and its buffering properties for stabilizing the pH of the composition at a pH from 6 to 8. The buffer, taken on its own, has a pH of at least 9.

Examples 1-90 disclose compositions comprising Mesna and a dye selected from the group consisting of indocyanine green, riboflavin, riboflavin 5′-phosphate sodium, methylene blue and fluorescein. All these dyes are accepted for parenteral use in humans.

Indocyanine green is a fluorescent cyanine dye with a peak spectral absorption at about 800 nm, i.e. it is a green-colored dye. For use in combination with Mesna, indocyanine green shows the advantage of acting as a reductant, thus not reacting with Mesna which is prone to oxidation. Besides, the green color has the advantage of being especially visible on tissues.

As a fluorescent dye, riboflavin provides a yellow to orange-yellow coloring to objects stained with it. For use in combination with Mesna, riboflavin shows the advantage of acting as a reductant, thus not reacting with Mesna which is prone to oxidation.

Riboflavin 5′-phosphate sodium is the phosphate sodium salt of riboflavin. Like riboflavin, riboflavin 5′-phosphate sodium provides a yellow to orange-yellow coloring to objects stained with it. Like riboflavin, for use in combination with Mesna, riboflavin 5′-phosphate shows the advantage of acting as a reductant, thus not reacting with Mesna which is prone to oxidation.

Methylene blue is a fluorescent thiazine dye with a blue color. Methylene blue acts as an oxidant, yet in the relative amounts of methylene blue in respect of Mesna in Examples 10-12, Examples 25-27, Examples 40-42, Examples 55-57, Examples 70-72 and Examples 85-87 below, oxidation of Mesna by methylene blue is negligible.

Fluorescein is a red-colored fluorophore which appears purple under UV light. When applying UV light, fluorescein is thus well-suited for visualizing a composition comprising Mesna on tissues.

In Examples 1-30 and 61-75, the selected dyes are comprised in the compositions from 0.01 weight per volume percent to 0.03 weight per volume percent and in Examples 31-60 and 76-90, selected dyes are comprised in the compositions from 0.04 weight per volume percent to 0.06 weight per volume percent. Said concentrations of the one or more dyes are large enough to allow sufficient staining and clear visualization of tissues on which a composition comprising Mesna and one or more dyes is applied while not too large so that excessive use or wastage of the one or more dyes occurs or that undesired damage to the tissues would take place because of the one or more dyes. The staining of tissues with the one or more dyes functions to visualize parts of the tissues on which the Mesna composition is applied. This entails the advantage that a medical practitioner can see the parts where the composition is applied, allowing intuitive and quick ascertaining of a desired application of Mesna to the tissues, or if the application of Mesna should be adjusted, which adjustments can also be directly seen by the visualization brought about by the one or more dyes. The use of a dye allows to visualize whether the mesna solution remains in place for a sufficient duration for effective chemical action. It also helps visualize the cleavage plane, which is very useful to the surgeon. This is of great importance for allowing efficient dissection of tissues facilitated by Mesna, and certainly in view of the knowledge that flooding a surgical cavity in order to make sure that all tissues to be dissected are wetted by Mesna is not an option, because the amount of Mesna which can be used in a single surgical procedure is limited by toxicity considerations.

TABLE 1
Compositions of Examples (Ex.) 1-15 with specific concentrations of Mesna and selected dyes
solubilized in a volume of water, the concentrations expressed in weight per volume percent
(w/v %) with respect to the volume of water, according to embodiments of the invention

| | Ex. 1 | Ex. 2 | Ex. 3 | Ex. 4 | Ex. 5 | Ex. 6 | Ex. 7 | Ex. 8 |
|---|---|---|---|---|---|---|---|---|
| Mesna (w/v %) | 2-6 | 8-12 | 18-22 | 2-6 | 8-12 | 18-22 | 2-6 | 8-12 |
| indocyanine | 0.01-0.03 | 0.01-0.03 | 0.01-0.03 | | | | | |
| green (w/v %) | | | | | | | | |
| riboflavin | | | | 0.01-0.03 | 0.01-0.03 | 0.01-0.03 | | |
| (w/v %) | | | | | | | | |
| riboflavin | | | | | | | 0.01-0.03 | 0.01-0.03 |
| 5′-phosphate | | | | | | | | |
| sodium (w/v % | | | | | | | | |
| methylene blue | | | | | | | | |
| (w/v %) | | | | | | | | |
| fluorescein | | | | | | | | |
| (w/v %) | | | | | | | | |

| | Ex. 9 | Ex. 10 | Ex. 11 | Ex. 12 | Ex. 13 | Ex. 14 | Ex. 15 |
|---|---|---|---|---|---|---|---|
| Mesna (w/v %) | 18-22 | 2-6 | 8-12 | 18-22 | 2-6 | 8-12 | 18-22 |
| indocyanine | | | | | | | |
| green (w/v %) | | | | | | | |
| riboflavin | | | | | | | |
| (w/v %) | | | | | | | |
| riboflavin | 0.01-0.03 | | | | | | |
| 5′-phosphate | | | | | | | |
| sodium (w/v % | | | | | | | |
| methylene blue | | 0.01-0.03 | 0.01-0.03 | 0.01-0.03 | | | |
| (w/v %) | | | | | | | |
| fluorescein | | | | | 0.01-0.03 | 0.01-0.03 | 0.01-0.03 |
| (w/v %) | | | | | | | |

TABLE 2
Compositions of Examples (Ex.) 16-30 with specific concentrations of Mesna and selected dyes solubilized
in 10 mM Na₂HPO₄ aqueous solution, the concentrations expressed in weight per volume percent (w/V %)
with respect to the volume of the 10 mM Na₂HPO₄ aqueous solution, according to embodiments of the invention

| | Ex. 16 | Ex. 17 | Ex. 18 | Ex. 19 | Ex. 20 | Ex. 21 | Ex. 22 | Ex. 23 |
|---|---|---|---|---|---|---|---|---|
| Mesna (w/v %) | 2-6 | 8-12 | 18-22 | 2-6 | 8-12 | 18-22 | 2-6 | 8-12 |
| indocyanine | 0.01-0.03 | 0.01-0.03 | 0.01-0.03 | | | | | |
| green (w/v %) | | | | | | | | |
| riboflavin | | | | 0.01-0.03 | 0.01-0.03 | 0.01-0.03 | | |
| (w/v %) | | | | | | | | |
| riboflavin | | | | | | | 0.01-0.03 | 0.01-0.03 |
| 5′-phosphate | | | | | | | | |
| sodium (w/v %) | | | | | | | | |
| methylene blue | | | | | | | | |
| (w/v %) | | | | | | | | |
| fluorescein | | | | | | | | |
| (w/v %) | | | | | | | | |

| | Ex. 24 | Ex. 25 | Ex. 26 | Ex. 27 | Ex. 28 | Ex. 29 | Ex. 30 |
|---|---|---|---|---|---|---|---|
| Mesna (w/v %) | 18-22 | 2-6 | 8-12 | 18-22 | 2-6 | 8-12 | 18-22 |
| indocyanine | | | | | | | |
| green (w/v %) | | | | | | | |
| riboflavin | | | | | | | |
| (w/v %) | | | | | | | |
| riboflavin | 0.01-0.03 | | | | | | |
| 5′-phosphate | | | | | | | |
| sodium (w/v %) | | | | | | | |
| methylene blue | | 0.01-0.03 | 0.01-0.03 | 0.01-0.03 | | | |
| (w/v %) | | | | | | | |
| fluorescein | | | | | 0.01-0.03 | 0.01-0.03 | 0.01-0.03 |
| (w/v %) | | | | | | | |

TABLE 3
Compositions of Examples (Ex.) 31-45 with specific concentrations of Mesna and selected
dyes solubilized in water, the concentrations expressed in weight per volume percent
(w/V %) with respect to the volume of water, according to embodiments of the invention

| | Ex. 31 | Ex. 32 | Ex. 33 | Ex. 34 | Ex. 35 | Ex. 36 | Ex. 37 | Ex. 38 |
|---|---|---|---|---|---|---|---|---|
| Mesna (w/v %) | 2-6 | 8-12 | 18-22 | 2-6 | 8-12 | 18-22 | 2-6 | 8-12 |
| indocyanine | 0.04-0.06 | 0.04-0.06 | 0.04-0.06 | | | | | |
| green (w/v %) | | | | | | | | |
| riboflavin | | | | 0.04-0.06 | 0.04-0.06 | 0.04-0.06 | | |
| (w/v %) | | | | | | | | |
| riboflavin | | | | | | | 0.04-0.06 | 0.04-0.06 |
| 5′-phosphate | | | | | | | | |
| sodium (w/v %) | | | | | | | | |
| methylene blue | | | | | | | | |
| (w/v %) | | | | | | | | |
| fluorescein | | | | | | | | |
| (w/v %) | | | | | | | | |

| | Ex. 39 | Ex. 40 | Ex. 41 | Ex. 42 | Ex. 43 | Ex. 44 | Ex. 45 |
|---|---|---|---|---|---|---|---|
| Mesna (w/v %) | 18-22 | 2-6 | 8-12 | 18-22 | 2-6 | 8-12 | 18-22 |
| indocyanine | | | | | | | |
| green (w/v %) | | | | | | | |
| riboflavin | | | | | | | |
| (w/v %) | | | | | | | |
| riboflavin | 0.04-0.06 | | | | | | |
| 5′-phosphate | | | | | | | |
| sodium (w/v %) | | | | | | | |
| methylene blue | | 0.04-0.06 | 0.04-0.06 | 0.04-0.06 | | | |
| (w/v %) | | | | | | | |
| fluorescein | | | | | 0.04-0.06 | 0.04-0.06 | 0.04-0.06 |
| (w/v %) | | | | | | | |

TABLE 4
Compositions of Examples (Ex.) 46-60 with specific concentrations of Mesna and selected dyes in a
10 mM Na₂HPO₄ aqueous solution, the concentrations expressed in weight per volume percent (w/v %)
with respect to the volume of the 10 mM Na₂HPO₄ aqueous solution, according to embodiments of the invention

| | Ex. 46 | Ex. 47 | Ex. 48 | Ex. 49 | Ex. 50 | Ex. 51 | Ex. 52 | Ex. 53 |
|---|---|---|---|---|---|---|---|---|
| Mesna (w/v %) | 2-6 | 8-12 | 18-22 | 2-6 | 8-12 | 18-22 | 2-6 | 8-12 |
| indocyanine | 0.04-0.06 | 0.04-0.06 | 0.04-0.06 | | | | | |
| green (w/v %) | | | | | | | | |
| riboflavin | | | | 0.04-0.06 | 0.04-0.06 | 0.04-0.06 | | |
| (w/v %) | | | | | | | | |
| riboflavin | | | | | | | 0.04-0.06 | 0.04-0.06 |
| 5′-phosphate | | | | | | | | |
| sodium (w/v %) | | | | | | | | |
| methylene blue | | | | | | | | |
| (w/v %) | | | | | | | | |
| fluorescein | | | | | | | | |
| (w/v %) | | | | | | | | |

| | Ex. 54 | Ex. 55 | Ex. 56 | Ex. 57 | Ex. 58 | Ex. 59 | Ex. 60 |
|---|---|---|---|---|---|---|---|
| Mesna (w/v %) | 18-22 | 2-6 | 8-12 | 18-22 | 2-6 | 8-12 | 18-22 |
| indocyanine | | | | | | | |
| green (w/v %) | | | | | | | |
| riboflavin | | | | | | | |
| (w/v %) | | | | | | | |
| riboflavin | 0.04-0.06 | | | | | | |
| 5′-phosphate | | | | | | | |
| sodium (w/v %) | | | | | | | |
| methylene blue | | 0.04-0.06 | 0.04-0.06 | 0.04-0.06 | | | |
| (w/v %) | | | | | | | |
| fluorescein | | | | | 0.04-0.06 | 0.04-0.06 | 0.04-0.06 |
| (w/v %) | | | | | | | |

### Example 91: Stability Study of Solutions Comprising Mesna and Riboflavin 5′-Phosphate Sodium

A stability study was conducted for the purpose of evaluating a possible interaction between riboflavin 5′-phosphate sodium, a dye accepted for parenteral use in humans, and Mesna.

The following protocol was followed:
30 ml of phosphate buffer was added to Mesna powder for reconstitution, resulting in a solution comprising Mesna;
riboflavin 5′-phosphate sodium was then added at 7 and 15 mg to obtain 2 different concentrations, namely 0.23 mg/mL and 0.5 mg/mL;
each solution was then placed at 4° C. and 20° C. during 1 hour and 24 hours before analysis;
HPLC analyses according to the recommendations of the European Pharmacopoeia 9th edition were performed (“R” refers to Pharmacopoeia reagents):
   Standards of Mesna: 10.0-5.0-2.0-1.0 mg/mL
   Volume taken: 1 mL diluted in 10 mL of mobile phase
   Mobile phase:
      2.94 g of potassium dihydrogenphosphate
      2.94 g of dipotassium hydrogen phosphate R
      2.6 g hydrogen sulfate tetrabutylammonium R
      Ad 600 mL of water R
      Adjust pH to 2.3 with phosphoric acid
      Add 335 mL of R-methanol
      Ad 1000 mL of water R
   Flow rate: 1 mL/min
   Detection: 235 nm
   Injection volume: 20 μL
   Run time: 18 minutes
   Number of analyzes per sample: 1

The first step was to verify the absence of interference between the peak corresponding to the Mesna and that corresponding to riboflavin 5′-phosphate sodium using the assay method/Mesna analysis conditions described in European Pharmacopoeia 9th edition and included in the protocol above.

Using the assay/assay method of assaying Mesna and related substances described in European Pharmacopoeia 9th edition, detection of riboflavin 5′-phosphate sodium did not interfere with that of Mesna. The latter can therefore be quantified without risk of interference with the selected dye.

Staining of the Mesna solution after addition of riboflavin 5′-phosphate sodium, whatever the concentration of riboflavin 5′-phosphate sodium, turned the solution from colorless to translucent yellow.

In order to validate the obtained data, a reference corresponding to a reconstituted solution of Mesna without riboflavin 5′-phosphate sodium was analyzed at the same time as the samples comprising riboflavin 5′-phosphate sodium (Table 5).

TABLE 5
Concentrations of Mesna in a phosphate buffer, without
and with (0.23 and 0.50 mg/mL) riboflavin 5′-phosphate
sodium (RF5′PNa), immediately after reconstitution
and after 1 h and 24 h, at 4° C. and 20° C.

| | 0 h | 1 h | | 24 h | |
|---|---|---|---|---|---|
| | mg/ml | mg/ml | % variation* | mg/ml | % variation* |
| 4° C. | | | | | |
| Reference | 4.75 | 4.78 | +0.63 | 4.89 | +2.95 |
| +0.23 mg/ml | 4.48 | 4.51 | +0.67 | 4.62 | +3.12 |
| RF5′PNa | | | | | |
| +0.50 mg/ml | 4.59 | 4.62 | +0.65 | 4.67 | +1.74 |
| RF5′PNa | | | | | |
| 20° C. | | | | | |
| Reference | 4.65 | 4.67 | +0.43 | 4.77 | +2.58 |
| +0.23 mg/ml | 4.47 | 4.50 | +0.67 | 4.87 | +8.95 |
| RF5′PNa | | | | | |
| +0.50 mg/ml | 4.57 | 4.61 | +0.88 | 4.62 | +1.09 |
| RF5′PNa | | | | | |

*The percentage variation is calculated relative to the reference under the same storage conditions

Under the analytical conditions described by the European Pharmacopoeia 9th edition for the quantification of Mesna and its related substances, the variations in Mesna content do not increase by more than 1% after addition of riboflavin 5′-phosphate sodium, whatever either its concentration in the device (0.23 mg/mL and 0.50 mg/mL) and the storage temperature (4° C. and 20° C.). The increases evaluated are in line with those observed at the reference level.

After 24 hours, the percentages of variation increase significantly. However, relative to the reference not comprising riboflavin 5′-phosphate sodium, the increases in levels did not seem significant, except for the sample comprising 0.23 mg/mL riboflavin 5′-phosphate sodium placed at 20° C.

In conclusion for Example 91, it can be concluded that the riboflavin 5′-phosphate sodium does not interfere with or degrade Mesna under the storage conditions described.

### Example 92: Stability Study of Mesna Powder in the Presence of Riboflavin 5′-Phosphate Sodium

About 1.5 g of Mesna powder was accurately weighed, without or with 7 mg or 15 mg of riboflavin 5′-phosphate sodium, and placed in a dry oven at 20° C. for 1 month to test its stability (Table 6).

TABLE 6
Results of stability tests on Mesna powder in the absence and presence
of riboflavin 5′-phosphate sodium (RF5′PNa) at 20° C.

| | | 1.5 g Mesna powder + 7 mg | | 1.5 g Mesna powder + 15 mg | |
|---|---|---|---|---|---|
| 1.5 g Mesna powder | | RF5′PNa | | RF5′PNa | |
| W0** (g) | W1*** (g) | W0** (g) | W1*** (g) | W0** (g) | W1*** (g) |
| 4.907 ± 0.038* | 4.807 ± 0.016* | 4.887 ± 0.041* | 4.801 ± 0.003* | 4.900 ± 0.038* | 4.827 ± 0.002* |

*standard deviation
**W0 = weight before placement in dry oven at 20° C.
***W1 = weight after 1 month in dry oven at 20° C.

Given the standard deviations, it can concluded that Mesna is stable in the presence of riboflavin 5′-phosphate sodium, in dry form, for 1 month at 20° C.

### Examples 93-107: Compositions Comprising Mesna for Use in a Method of Robotic Assisted Surgery, According to Embodiments of the Invention

Examples 93-107 concern compositions with different concentrations of Mesna ranging from 4% by weight to 22% by weight, according to embodiments of the invention. The compositions according to Examples 93-107, shown in Table 7, are optimally suited for use in a method of robotic assisted surgery.

When used in a method of robotic assisted surgery, weight percentages of Mesna of 4% by weight to 22% by weight in the composition, as shown in Examples 93-107, are high enough for facilitating tissue separation and are low enough to avoid excessive wastage of Mesna in a body of a patient where not necessary, thereby not exceeding a maximum amount of Mesna acceptable to prevent systemic potential toxicity.

Water (cf. Examples 93-95 and 102-107), in combination with NaCl (cf. Examples 102-107) to be construed as physiological serum, owing to its pH and solubilizing properties for solubilizing Mesna, is desired as a pharmaceutically acceptable solvent in the present context.

10 mM Na₂HPO₄ (aqueous) solution (cf. Examples 96-98) and a 10 mM Na₂HPO₄ and 75 mM NaCl (aqueous) solution (cf. Examples 99-101) are desired pharmaceutically acceptable buffers in the present context, owing to their solubilizing properties for solubilizing Mesna and their buffering properties for stabilizing the pH of the composition at a pH from 6 to 8. The two aforementioned pharmaceutically acceptable buffers, taken on their own, have a pH of at least 9.

Disodium edetate is added as a chelating agent in Examples 102-107 for stabilization purposes. Sodium hydroxide is added in Examples 102-107 for pH adjustment purposes.

TABLE 7
Compositions of Examples (Ex.) 93-107 with different concentrations of Mesna ranging
from from 4% by weight to 22% by weight, according to embodiments of the invention

| | Ex. 93 | Ex. 94 | Ex. 95 | Ex. 96 | Ex. 97 | Ex. 98 | Ex. 99 | Ex. 100 | Ex. 101 | Ex. 102 |
|---|---|---|---|---|---|---|---|---|---|---|
| Mesna | 4-6 | 8-12 | 18-22 | 4-6 | 8-12 | 18-22 | 4-6 | 8-12 | 18-22 | 4-6 |
| (% by weight) | | | | | | | | | | |
| water | 94-96 | 88-92 | 78-82 | | | | | | | 92.9-95.3 |
| (% by weight) | | | | | | | | | | |
| 10 mM | | | | 94-96 | 88-92 | 78-82 | | | | |
| Na₂HPO₄ | | | | | | | | | | |
| solution | | | | | | | | | | |
| (% by weight) | | | | | | | | | | |
| 10 mM | | | | | | | 94-96 | 88-92 | 78-82 | |
| Na₂HPO₄ | | | | | | | | | | |
| and 75 mM | | | | | | | | | | |
| NaCl solution | | | | | | | | | | |
| (% by weight) | | | | | | | | | | |
| NaCl | | | | | | | | | | 0.7-1.1 |
| (% by weight) | | | | | | | | | | |
| disodium | | | | | | | | | | |
| edetate | | | | | | | | | | |
| (% by weight) | | | | | | | | | | |
| sodium | | | | | | | | | | |
| hydroxide | | | | | | | | | | |
| (% by weight) | | | | | | | | | | |

| | Ex. 103 | Ex. 104 | Ex. 105 | Ex. 106 | Ex. 107 |
|---|---|---|---|---|---|
| Mesna | 8-12 | 18-22 | 4-6 | 8-12 | 18-22 |
| (% by weight) | | | | | |
| water | 86.8-91.6 | 76.9-81.7 | 92.3-94.8 | 86.4-93.0 | 76.7-81.2 |
| (% by weight) | | | | | |
| 10 mM | | | | | |
| Na₂HPO₄ | | | | | |
| solution | | | | | |
| (% by weight) | | | | | |
| 10 mM | | | | | |
| Na₂HPO₄ | | | | | |
| and 75 mM | | | | | |
| NaCl solution | | | | | |
| (% by weight) | | | | | |
| NaCl | 0.4-1.2 | 0.3-1.1 | 0.35-0.55 | 0.3-0.5 | 0.2-0.4 |
| (% by weight) | | | | | |
| disodium | | | 0.8-1.0 | 0.75-0.95 | 0.6-0.8 |
| edetate | | | | | |
| (% by weight) | | | | | |
| sodium | | | 0.05-0.15 | 0.02-0.12 | 0.01-0.1 |
| hydroxide | | | | | |
| (% by weight) | | | | | |

### Examples 108-111: Methods for Producing a Composition Comprising Mesna for Use in a Method of Robotic Assisted Surgery, According to Embodiments of the Invention

The methods according to Examples 108-111 are performed at most one day prior to performing a method of robotic assisted surgery.

According to Example 108, one first volume of a Mesna solution comprising 100 mg/mL Mesna solubilized in a mixture of disodium edetate, sodium hydroxide and sterile water was mixed with a second equal volume of sterile water comprising 0.9 mass/volume percent sodium chloride, resulting a composition comprising 5% by weight of solubilized Mesna.

According to Examples 109-111, sterile Mesna in powder form is solubilized in a volume of water, preferably in a volume of sterile water, in order to produce compositions comprising 4-6% by weight of Mesna and 94-96% by weight of water, 8-12% by weight of Mesna and 88-92% by weight of water, or 18-22% by weight of Mesna and 78-82% by weight of water, respectively.

The measure of preparing the composition by dissolving Mesna in powder form in water has the advantages that Mesna oxidation is avoided and that compositions comprising tailor-made concentrations of Mesna and volumes of the composition comprising Mesna can be prepared case-by-case in function of the needs for a method of robotic assisted surgery.

One skilled in the art can be assumed to appreciate that more examples of methods for producing a composition comprising Mesna for use in a method of robotic assisted surgery are covered by the present teachings, as for example methods resulting in the compositions according to Examples 93-107 described above.

### Example 112: A Robot Instrument for Chemically Assisted Mechanical Dissection According to the Invention

In FIGS. 11-15 the preferred embodiment of the robot instrument for chemically assisted mechanical dissection of the invention are disclosed.

The general arrangement of the components for chemically assisted surgery with robots is shown on FIG. 11.

The source of composition comprising Mesna is obtained from the cartridge 401. The cartridge contains Mesna in solid form apart from a solvent. The Mesna is dissolved in the solvent by a rupture of a separating membrane preferably less than 12 hours before use.

The fluid connection between the cartridge 401 and the robot arm 402 is made by a disposable tubing 403, with male Luer lock connectors at both ends.

In a special embodiment, the female Luer connection of the cartridge 404 includes a normally closed valve which open upon connection of the male Luer from the tubing.

In a preferred embodiment, a particle filter 405 is inserted at the inlet side of the tubing 403. A disposable peristaltic pump cassette 406 is inserted in the tubing. The cassette is attached to the pump motor 407 before use. The motor is turned on when the foot pedal 408 which is under the foot of an operator, seated behind a console 430. The outlet of the tubing is connected to the gearbox of the robot arm 402.

In a preferred embodiment, the pump motor 407 can be attached to a pole 410 or laid down on a flat surface near the robot. In a preferred embodiment, the cartridge 401 is hung on a hanger attached to the pump motor 407.

FIG. 12 represents a robot arm for chemically assisted dissection intended for laparoscopic procedures.

The arm is composed of an irrigated tip 411 described in more details below, a shaft 412 whereby the irrigation tip is mounted on and housing the cables for moving the irrigation tip and the fluid channel 414 for dispensing the Mesna composition by the irrigation tip and a gear box 402 housing the pulleys connected to the driver motors. The gear box 402 is also fitted with a Luer connection for the supply of the Mesna composition.

The fluid channel is preferably provided by extruding a lumen 414 in the stainless-steel shaft in order to leave enough room for the cables between the gear box 402 and the irrigation tips 411. The irrigation tips 411 are capable of dispensing topically the Mesna composition on the tissues to be dissected while exerting a mechanical separation force, both controlled by the robot operator.

FIG. 13 describes a Maryland type of dissector tip. The jaws 415 and 416 can be moved independently by the robot operator and can be used either in pulling mode (forceps) or dissection mode.

At least one the jaws is fitted with an orifice 417 for dispensing the Mesna composition on the tissues to be dissected before exerting the mechanical separation force. The distal part 418 holds the jaws which can be rotated independently around the axis 419. The proximal part 420 is attached mechanically to the shaft. It holds the distal piece which can be rotated around the axis 421. The jaws are connected electrically to the metallic shaft by the metallic distal and proximal pieces. At least one of the jaws is connected fluidly to the fluid channel provided in the shaft through the distal and proximal pieces.

The provision of the fluid connection to the jaws is described on FIGS. 14 and 15. A fluid channel 413 is provided in the proximal piece 420 in front of the outlet of the channel 414 for tight fluid connection. A groove 422 is provided in the pulley 423 provided in the proximal piece which is rotating the distal piece around its axis. The fluid channel 413 in the proximal piece has its orifice 424 in front of the groove.

A fluid channel 425 is also provided in the distal piece 418. The proximal orifice of this channel is located in front of the groove 422. In this way, the fluid can pass from the proximal piece 420 to the distal piece 418. Fluid tightness is obtained by the flat flanges on the pulleys and the pieces which are closing the groove 422.

Similarly, a groove 426 is provided in the basis of the irrigated jaw 415. The distal orifice 428 of the fluid channel 425 provided in the distal piece is placed in front of the groove 426. The proximal orifice 429 of the fluid channel provided in the irrigated jaw 415 is also located in front of the groove 426. Fluid tightness is obtained by the flat flanges on the jaw basis and the distal piece. In this way, the dispensing of the Mesna composition is made at the orifice 417 of one of the jaws. The control of the dispensing is achieved by the robot operator by depressing the foot pedal 408.

Although the present invention has been described with reference to preferred embodiments thereof, many modifications and alternations may be made by a person having ordinary skill in the art without departing from the scope of this invention which is defined by the appended claims.

What is claimed is:

1. A composition comprising sodium 2-mercaptoethanesulfonate (Mesna), and one or more dyes.

2. The composition according to claim 1, wherein the one or more dyes is selected from the group consisting of indocyanine green, riboflavin, riboflavin 5′-phosphate sodium, methylene blue and fluorescein.

3. The composition according to claim 1, wherein the composition comprises one or more pharmaceutically acceptable solvents and/or one or more pharmaceutically acceptable buffers, the one or more solvents and/or one or more buffers comprising from 0.2 weight per volume percent to 30 weight per volume percent of Mesna and from 0.0001 weight per volume percent to 30 weight per volume percent of the one or more dyes.

4. The composition according to claim 3, wherein the one or more pharmaceutically acceptable solvents is selected from the group comprising water and a solution of up to 0.9 mass/volume percent of NaCl in water.

5. The composition according to claim 3, wherein the pharmaceutically acceptable buffer is a solution of Na₂HPO₄ in water or a solution of Na₂HPO₄ and NaCl in water.

6. A method of surgery comprising separating tissue layers along a cleavage plane between said tissue layers, wherein the method comprises applying the composition according to claim 1 to said cleavage plane.

7. A method for visualizing a composition comprising Mesna which is applied to tissues, comprising applying the composition to two or more tissues, wherein the composition comprises one or more dyes for visualizing parts of the two or more tissues where the composition is applied.

8. The method according to claim 7, wherein the one or more dyes is selected from the group consisting of indocyanine green, riboflavin, riboflavin 5′-phosphate sodium, methylene blue and fluorescein.

9. The method according to claim 7, wherein the one or more dyes is one or more fluorescent dyes and wherein the method further comprises providing a fluorescence surgical goggle to enable visualization of the fluorescent dyes through the goggle.

10. A robot instrument for chemically assisted mechanical dissection comprising:
a cartridge comprising a composition comprising Mesna in a liquid form;
a pump connected to said cartridge to feed the composition comprising Mesna to an outlet tubing of said pump upon actuation of said pump;
a robot arm comprising an irrigation channel with an inlet connected to said outlet tubing of said pump and comprising an outlet connected to an irrigation tip mounted on said robot arm, wherein said tip comprises at least one surgical tool selected from the group comprising scalpels, scissors, bovies, forceps, dissector, elevators, hooks, probes, needles, knot pushers, retractors, scopes, clamps and graspers, and wherein the tip further comprises at least one irrigation orifice on said at least one surgical tool, said irrigation orifice fluidly connected to the irrigation channel,
whereby actuation of the pump supplies the composition comprising Mesna from the cartridge through the irrigation channel to said irrigation tip of the mounted robot arm, and whereby the actuation of the pump is controllable by an operating switch.

11. The robot instrument according to claim 10, wherein said robot arm and feeding of the Mesna solution is steerable by a computer-assisted electromechanical device by a robot operator.

12. The robot instrument according to claim 10 wherein said robot instrument is equipped by a haptic feedback to an operator.

13. The robot instrument according to claim 10, wherein the cartridge further comprises at least one dye.

14. The robot instrument according to claim 10, whereby a speed of said irrigation is controllable by a pump.

15. The robot instrument according to claim 10, wherein a diameter of the robot tip orifice is in range of 100 μm to 500 μm.

16. The robot instrument according to claim 10, wherein the robot arm tip comprises one or more conductive materials connected to a radio frequency source which can be operated in an electric mode for cutting or cauterizing without changing instruments.
